(19) United States
(12) Patent Application Publication
(10) Pub. No.: US 2019/0391142 A1
(43) Pub. Date: Dec. 26, 2019

(54) SURFACE AND DIFFUSION ENHANCED BIOSENSOR

(71) Applicant: PLEXENSE, INC., Davis, CA (US)

(72) Inventors: Jinwoo Jeon, Seoul (KR); Hyejin Hwang, Yongin-si (KR); Yeon-Su Park, Yuseong-gu (KR); Gibum Kim, Sacramento, CA (US)

(21) Appl. No.: 16/390,748

(22) Filed: Apr. 22, 2019

Related U.S. Application Data
(63) Continuation-in-part of application No. PCT/KR2017/014013, filed on Dec. 1, 2017.
Application No. PCT/KR2017/014013 is a continuation-in-part of application No. PCT/KR2017/011520, filed on Oct. 18, 2017.
(60) Provisional application No. 62/662,088, filed on Apr. 24, 2018.

(30) Foreign Application Priority Data
Oct. 20, 2016 (KR) 10-2016-0136342
Dec. 2, 2016 (KR) 10-2016-0163521

(51) Int. Cl. G01N 33/543; B01L 3/00

(52) U.S. Cl. CPC G01N 33/54366; B01L 3/50853; B01L 2300/0832; B01L 2300/042; B01L 2300/0636; B01L 2300/046; B01L 3/523

(57) ABSTRACT

The disclosed technology generally relates to a biosensor configured for immunoassay, and more specifically to a biosensor having structures configured to enhance speed and sensitivity of immunoassay, and to test kits and methods of immunoassay using same. In one aspect, a sensor assembly adapted for an enzyme linked immunosorbent assay (ELISA) comprises a sensor strip comprising one or more wells formed therein. The sensor assembly additionally comprises one or more detection structures connected to a sidewall of each of the one or more wells, wherein the one or more detection structures are configured to immobilize a biological molecule directly thereon.

### INCORPORATION BY REFERENCE

This application claims the benefit of priority of U.S. Provisional Application No. U.S. 62/662,088, filed Apr. 24, 2018, is a continuation in part of PCT Application No. PCT/KR2017/014013, filed Dec. 1, 2017, which claims priority to Korean Patent Application No. 10-2016-0163521, filed Dec. 2, 2016, and is a continuation in part of PCT Application No. PCT/KR2017/011520, filed Oct. 18, 2017, which claims priority to Korean Patent Application No. 10-2016-0136342, filed Oct. 20, 2016. The content of each of the above applications is incorporated herein by reference in its entirety.

This application incorporates by reference PCT Application No. PCT/KR2017/004546, filed Apr. 28, 2017, which claims priority to Korean Patent Application No. 10-2016-0060161, filed May 17, 2016, and Korean Patent Application No. 10-2017-0171638, filed Dec. 13, 2017. The content of each of the above applications is incorporated herein by reference in its entirety.

### BACKGROUND

### Field

The disclosed technology generally relates to a biosensor configured for immunoassay techniques, and more specifically to a biosensor having structures configured to enhance speed and sensitivity of immunoassay techniques, and to test kits and methods of immunoassay techniques using same.

### Description of the Related Art

ELISA (enzyme-linked immunosorbent assay) is an assay technique for detecting and quantifying a target analyte, which can include substances such as peptides, proteins, antibodies and hormones. In an ELISA, the target analyte, e.g., an antigen, is immobilized on a solid surface and then complexed with a reagent, e.g., antibody, that is linked to an enzyme. Detection is accomplished by assessing the conjugated enzyme activity via incubation with a substrate to produce a detectable reaction product.

### SUMMARY

The disclosed embodiments are generally aimed at providing a biosensor assembly with enhanced sensitivity and faster detection of target analytes. The biosensor assemblies according to embodiments are constructed such that the concentration of immobilized reactant or antibody is increased, thereby increasing the speed of various ELISA techniques. The biosensor assemblies according to embodiments can also reduce the hook effect, a disadvantage in presently known one-step ELISA techniques. The disclosed embodiments are also aimed at providing a biosensor assembly that is very convenient to use and can greatly reduce the complexity and time (e.g., to less than 45 minutes) consumed for analysis compared to conventional immunoassay techniques.

In a first aspect, a sensor assembly adapted for an enzyme linked immunosorbent assay (ELISA) comprises a sensor strip comprising one or more wells formed therein. The sensor assembly additionally comprises one or more detection structures connected to a sidewall of each of the one or more wells, wherein the one or more detection structures are configured to immobilize a biological molecule directly thereon.

In a second aspect, a sensor assembly adapted for an ELISA comprises a cuvette comprising a cavity and a cap configured to close the cavity. The sensor assembly additionally includes one or more detection structures connected to the cap and configured to be at least partly immersed in a liquid sample when present in the cuvette, wherein the one or more detection structures are configured to immobilize a biological molecule directly thereon.

In a third aspect, an enzyme linked immunosorbent assay (ELISA) kit comprises one or more reagents for an ELISA and a sensor assembly adapted for the ELISA. The sensor assembly comprises container, e.g., a transparent container having at least one transparent surface, having one or more cavities formed therein, a plurality of active surfaces disposed in each of the one or more cavities and configured for immobilizing a reagent thereon, and one or more detection structures, e.g., transparent detection structures, disposed in each of the one or more cavities. Each of the transparent detection structures comprises one or more main surfaces that provide one or more of the active surfaces. The one or more cavities are configured to be filled with a liquid such that each of the transparent detection structures are at least partially submerged therein. A ratio of a combined surface area of the transparent structures contacted by the liquid to a volume of the liquid exceeds about 0.25 mm² per microliter. Each of the active surfaces is separated from an immediately adjacent one of the active surfaces by a distance exceeding about 500 microns.

In a fourth aspect, an enzyme linked immunosorbent assay (ELISA) kit comprises one or more reagents for an ELISA and a sensor assembly adapted for the ELISA. The sensor assembly comprises a transparent container having one or more cavities formed therein, a plurality of active surfaces disposed in each of the one or more cavities and configured for immobilizing a reagent thereon, and one or more transparent detection structures disposed in each of the one or more cavities, wherein each of the transparent detection structures comprises one or more main surface that provide one or more of the active surfaces. The one or more cavities are configured to be filled with a liquid such that each of the transparent detection structures are at least partially submerged therein. A ratio of a combined surface area of the transparent structures contacted by the liquid to a volume of the liquid is between about 0.25 mm² per microliter and about 8.0 mm² per microliter.

In a fifth aspect, an enzyme linked immunosorbent assay (ELISA) kit comprises one or more reagents for an ELISA and a sensor assembly adapted for the ELISA. The sensor assembly comprises a transparent container having one or more cavities formed therein, a plurality of active surfaces disposed in each of the one or more cavities and configured for immobilizing a reagent thereon, and one or more transparent detection structures disposed in each of the one or more cavities. Each of the transparent detection structures comprises one or more main surfaces that provide one or more of the active surfaces. Each of the active surfaces is separated from an immediately adjacent one of the active surfaces by a distance between about 500 microns and about 8 mm.

In a sixth aspect, an enzyme linked immunosorbent assay (ELISA) kit comprises one or more reagents for an ELISA and a sensor assembly adapted for the ELISA. The sensor assembly comprises a transparent container having one or more cavities formed therein, a plurality of active surfaces disposed in each of the one or more cavities and configured for immobilizing a reagent thereon, and one or more transparent detection structures disposed in each of the one or more cavities. Each of the transparent detection structures comprises one or more main surfaces that provide one or more of the active surfaces. At least one of the active surfaces comprises a textured polymeric surface having microstructures or nanostructures.

In a seventh aspect, an enzyme linked immunosorbent assay (ELISA) kit comprises one or more reagents for an ELISA and a sensor assembly adapted for the ELISA. The sensor assembly comprises a transparent container having one or more cavities formed therein, and one or more transparent detection structures disposed in each of the one or more cavities. Inner surfaces of the cavities and main surfaces of the one or more transparent detection structures provide thereon active surfaces configured for immobilizing a reagent configured to specifically bind to an analyte. The main surfaces of the transparent detection structures are configured such that, upon performing the ELISA, a detectable optical density corresponding to the analyte specifically bound to the immobilized reagent is increased without decreasing a rate of specifically binding the analyte to the immobilized reagents, relative to the sensor assembly without the one or more transparent detection structures.

In an eighth aspect, a method of conducting an enzyme linked immunosorbent assay (ELISA) comprises providing an ELISA kit according to any of the above embodiments and conducting an ELISA reaction within the optically transparent container. Conducting the ELISA reaction comprises: providing a solution comprising a target analyte and a marker-labeled detection reagent that is configured to specifically bind to the target analyte; immobilizing on the active surfaces of the sensor assembly a capturing reagent configured to specifically bind to a target analyte; at least partially immersing the active surfaces in the solution to cause the target analyte to be specifically bound to the capturing reagent and to the marker-labeled detection reagent; and detecting the target analyte specifically bound to the capturing reagent and to the marker-labeled detection reagent.

In a ninth aspect, a method of conducting an ELISA comprises providing an ELISA well, wherein the ELISA well comprises: a transparent container and more than one enhancement layer within the optically transparent container, wherein the more than one enhancement layer is configured to allow an antibody to be bound to it, wherein the more than one enhancement layer provides a ratio of a combined surface area of the more than one enhancement layer to a volume of the liquid is between about 0.25 mm² per microliter and about 8.0 mm² per microliter. The method additionally comprises conducting an ELISA with the optically transparent container, wherein only a single wash is involved in the ELISA.

In an tenth aspect, a biosensor according to the disclosed embodiments includes a detection structure in the shape of a plate having a first surface and a second surface opposite the first surface wherein an immobilized biomolecule specifically binding to a target analyte is arranged on at least one of the first and second surfaces.

In the biosensor according to the tenth aspect, microstructures or nanostructures in the form of projections are formed on at least one of the first and second surfaces of the detection structure and are attached with the immobilized biomolecule on the outer surface thereof.

In the biosensor according to the tenth aspect, the target analyte is selected from the group consisting of amino acids, peptides, polypeptides, proteins, glycoproteins, lipoproteins, nucleosides, nucleotides, oligonucleotides, nucleic acids, sugars, carbohydrates, oligosaccharides, polysaccharides, fatty acids, lipid, hormones, metabolites, cytokines, chemokines, receptors, neurotransmitters, antigens, allergens, antibodies, substrates, cofactors, inhibitors, drugs, pharmaceuticals, nutrients, prions, toxins, poisons, explosives, pesticides, chemical warfare agents, biohazardous agents, bacteria, viruses, radioisotopes, vitamins, heterocyclic aromatic compounds, carcinogens, mutagens, narcotics, amphetamines, barbiturates, hallucinogens, waste products, contaminants, and mixtures thereof.

In the biosensor according to the tenth aspect, the detection structure is inserted into and immersed in a cuvette accommodating a sample containing the target analyte such that the immobilized biomolecule reacts with the target analyte.

The biosensor according to the tenth aspect, the biosensor further includes a gripping member connected to one end of the detection structure and gripped by a user.

The biosensor according to the tenth aspect, the biosensor further includes a cap connecting the detection structure to the gripping member and releasably inserted into the inlet of the cuvette.

The biosensor according to the tenth aspect, the biosensor further includes a fixing member arranged on the outer surface of the cap and whose shape is changed to create resilience when the cap is inserted into the cuvette wherein the fixing member is brought into close contact with the inner circumferential surface of the cuvette by the resilience.

In the biosensor according to the tenth aspect, the detection structure is divided into an immersion portion immersed in the sample and a non-immersion portion having a narrow portion whose width is smaller than that of the immersion portion.

In the biosensor according to the tenth aspect, the narrow portion is recessed from at least one of both sides of the detection structure and extends along the lengthwise direction of the detection structure.

In the biosensor according to the tenth aspect, the detection structure is provided in plurality and the detection structures are spaced apart from and parallel to each other.

The biosensor according to the tenth aspect, the biosensor further includes a pair of guards facing each other through the detection structure to protect the detection structure.

The biosensor according to the tenth aspect, the biosensor further includes at least one sensor strip including a body with a predetermined length and a plurality of reaction chambers recessed from one surface of the body to accommodate a sample containing the target analyte wherein the detection structure is arranged in each of the reaction chambers.

The biosensor according to the tenth aspect, the biosensor further includes a fixing plate having a surface to which the sensor strip is detachably attached.

In the biosensor according to the tenth aspect, the detection structure is provided in plurality and the detection structures are vertically, e.g., in a depth direction, spaced apart from each other.

The biosensor according to the tenth aspect, the biosensor further includes sample injection holes recessed from one surface of the body so as to be in communication with the reaction chambers.

The biosensor according to the tenth aspect, the biosensor further includes an insertion protrusion protruding from one surface of the fixing plate wherein the body is recessed or perforated to form an insertion recess into which the insertion protrusion is inserted such that the sensor strip is attached to the fixing plate.

The biosensor according to the tenth aspect, the biosensor further includes a fixing protrusion spaced from the insertion protrusion and protruding from one surface of the fixing plate such that the insertion protrusion comes into contact with an inwardly recessed corner of one end of the body when inserted into the insertion hole.

The features and advantages according to the disclosed embodiments will become apparent from the following description with reference to the accompanying drawings.

The biosensor according to the disclosed embodiments is constructed such that the concentration of a receptor or antibody reacting per unit volume is increased. Due to this construction, the biosensor according to the disclosed embodiments offers convenience for one-step assay, significantly reduces the time required for analysis, and achieves further improved sensitivity.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1A is a schematic flow diagram showing sequences of steps in an enzyme linked immunosorbent assay (ELISA) based on sequential reactions;

FIG. 1B is a schematic flow diagram showing sequence of steps in a one-step ELISA based on simultaneous reactions;

FIGS. 2A to 2C are cross-sectional side views schematically illustrating detection structures of biosensors according to exemplary embodiments;

FIGS. 3A to 3E are perspective views schematically illustrating microstructures or nanostructures formed on biosensors according to exemplary embodiments;

FIG. 3F is a scanning electron microscope image of nanopillars formed on biosensors according to exemplary embodiments;

FIG. 4A is a schematic cross-sectional view of a cuvette-type biosensor assembly;

FIG. 4B is a schematic cross-sectional view of a cuvette-type biosensor assembly comprising detections structures according to exemplary embodiments;

FIG. 4C is a perspective view of a cuvette-type biosensor according to embodiments;

FIG. 5 illustrates front views of the biosensor assembly including the biosensor illustrated in FIG. 4C and a cuvette into which the biosensor is to be inserted, according to embodiments;

FIG. 6 is a side view illustrating a state in which the biosensor illustrated in FIG. 4C is inserted into a cuvette, according to embodiments;

FIG. 7 is a perspective view of a cuvette-type biosensor according to further embodiments;

FIG. 8 is a perspective view of a strip-type biosensor assembly according to embodiments;

FIG. 9 is a perspective view of a detection structure of a strip-type biosensor assembly according to embodiments;

FIG. 10 is a perspective view of a sensor strip of a strip-type biosensor assembly according to embodiments;

FIG. 11 is a perspective view of a strip-type biosensor assembly according to further embodiments;

FIGS. 12A and 12B are perspective views of a portion of a sensor strip of a strip-type biosensor assembly according to embodiments;

FIG. 12C are perspective views of component layers of the portion of the sensor strip illustrated in FIGS. 12A and 12B;

FIG. 12D illustrates a perspective view of a partially stacked portion of the sensor strip illustrated in FIGS. 12A and 12B;

FIGS. 12E-12G illustrate different portions of sensor strips of a strip-type biosensor assembly having different stack configurations according to embodiments;

FIG. 12H is an experimental absorbance graph obtained using a strip-type biosensor assembly according to embodiments;

FIG. 13A schematically illustrates top-down views of component layers of a sensor strip of a strip-type biosensor assembly according to embodiments;

FIGS. 13B to 13D illustrate photographs of perspective and top down views of the sensor strip illustrated in FIG. 13A;

FIG. 14A schematically illustrates top-down views of component layers of a sensor strip of a strip-type biosensor assembly according to embodiments;

FIGS. 14B to 14D illustrate photographs of perspective and top down views of the sensor strip illustrated in FIG. 14A;

FIG. 15 illustrates a perspective view of a sensor strip of a strip-type biosensor assembly according to embodiments;

FIGS. 16A and 16B are upper and lower perspective views, respectively, of component layers of a sensor strip of a strip-type biosensor assembly according to embodiments;

FIGS. 16C and 16D are detailed upper and lower perspective views, respectively, of the component layers of the sensor strip illustrated in FIGS. 16A and 16B;

FIGS. 17A and 17B are experimental measurements of absorbance versus assay time and absorbance versus concentration for an analyte (HE4), respectively, which were measured using a biosensor assembly, according to embodiments.

FIG. 18A illustrates a perspective view of a portion of a sensor strip of a strip-type biosensor assembly according to embodiments;

FIGS. 18B-18E illustrate top down views of portions of sensor strips of different strip-type biosensor assemblies according to embodiments;

FIG. 19A illustrates a perspective view of a portion of a sensor strip of a strip-type biosensor assembly according to embodiments;

FIGS. 19B-19F illustrate top down views of portions of sensor strips of different strip-type biosensor assemblies according to embodiments;

FIGS. 20A-20D illustrate perspective views of reaction chambers of sensor strips of different strip-type biosensor assemblies according to embodiments;

FIGS. 21A and 21B are calculated graphs of surface area contacted by a sample versus a diameter of reaction chambers for strip-type biosensor assemblies according to embodiments;

FIG. 22 is a flow diagram illustrating a method of performing an ELISA using a cuvette-type biosensor assembly according to embodiments;

FIG. 23 is a flow diagram illustrating a method of performing an ELISA using a strip-type biosensor assembly according to embodiments;

FIGS. 24A to 24F are experimental measurements of absorbances of reaction products with various analytes at different concentrations, which were measured using a biosensor assembly according to embodiments; and

FIGS. 25A and 25B are experimental measurements of absorbances of reaction products with various analytes at different concentrations, which were measured using a biosensor assembly having different numbers of detection structures, according to embodiments.

### DETAILED DESCRIPTION

Other objects, advantages, and novel features of the invention will become more apparent from the following detailed description and preferred embodiments with reference to the appended drawings. In the drawings, the same elements are denoted by the same reference numerals even though they are depicted in different drawings. In the description of the disclosed technology, detailed explanations of related art are omitted when it is deemed that they may unnecessarily obscure the essence of the disclosed technology.

Various ELISA techniques using conventional sensor assemblies include multiple steps including washing and/or incubation, and take a long time to complete, e.g., >120 min. Thus, there is a need for a biosensor assembly that can enable simpler and faster ELISA techniques.

Regardless of the type of various ELISA techniques described above, a common challenge for faster and more sensitive ELISA involves increasing the signal of the detected target analyte while having relatively high reaction rates. Various embodiments of the disclosed technology address these and other challenges, which will now be described in detail with reference to the accompanying drawings.

Reactivity in immunoassay is determined by many factors. Particularly, the concentration of a protein in a sample participating in a reaction and the concentration of a receptor or antibody reacting with the protein are considered the most important factors.

For general ELISA, the reaction area of a microtiter plate for immunoassay is limited to the area including a sample, limiting the concentration of a receptor or antibody participating in a reaction per the volume of the same sample. However, the inventors have recognized that, some techniques of increasing the surface to volume ratio in an attempt to increase sensitivity can reduce diffusion of various reagents and/or the analyte in the ELISA, leading to longer reaction times. Various embodiments described herein address these and other competing needs to improve the overall sensitivity while simultaneously reducing the reaction times.

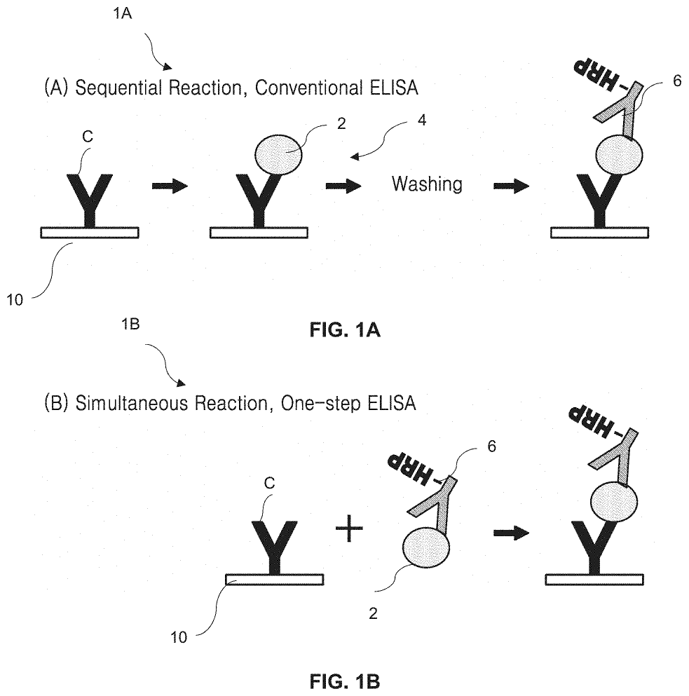

FIGS. 1A and 1B illustrate a trade-off between speed and accuracy observed in ELISA techniques. For illustrative purposes only, the trade-off is described in the context of a sandwich ELISA. However, analogous tradeoffs can be present in the context of other ELISA techniques. Referring to FIG. 1A, a general sandwich ELISA process 1A is illustrated. In the ELISA process 1A, a sample containing a target analyte 2, e.g., a protein or an antigen, is reacted with a receptor or capture antibody C immobilized on a substrate 10. For example, the substrate 10 can be a detection structure or an ELISA well plate. Subsequently, the reaction mixture is washed, and the reaction product 4 is reacted with a marker-labeled detection antibody 6. In the illustrated embodiment, the detection antibody 6 is conjugated to an enzyme, e.g., horseradish peroxidase (HRP), either directly or indirectly. Due to the complexity of the sequential reactions, ELISA techniques such as the illustrated sandwich ELISA technique is generally performed by a skilled user in laboratory and can last as long as 4 hours more to complete.

Referring to FIG. 1B, a modified sandwich ELISA process 1B is illustrated. In the ELISA process 1B, to reduce the time and complexity of the sequential sandwich ELISA illustrated in FIG. 1A, a marker-labeled detection antibody 6 is first mixed with a sample containing a target analyte 2, e.g., a protein or an antigen, and the mixture is allowed to react. The reaction mixture is reacted with a receptor or capture antibody C immobilized on a substrate 10. When the receptor or capture antibody C is immobilized on the substrate 10 prior to the reaction, the immunoassay can be performed by one-time injection of the sample without involving complicated analysis processes, which can potentially make the technique more convenient and shorten the time for analysis to about one-eighth or shorter compared to that of some conventional techniques. However, these benefits are often unrealized for various reasons. For example, when a large amount of the target protein is present, a portion of the target protein remains unreacted with the marker-labeled detection antibody and may react with the antibody immobilized on the substrate. This phenomenon is called the “hook effect.” Further, the presence of the unlabeled target protein makes it impossible to accurately determine the concentration of the target protein. These problems can be mitigated or solved when the concentration of the receptor or capture antibody participating in the reactions can be made higher than that of the target protein. Thus, there is a need for sensor assemblies adapted for ELISA techniques, e.g., a sandwich ELISA technique, that is faster, more sensitive and/or less complex.

### Sensor Assemblies Having Microstructured or Nanostructured Active Surfaces for Enhanced Sensitivity

As described herein, a biomolecule refers to any organic compound or a reagent that may participate in an immunoassay, e.g., ELISA, directly or indirectly, including antibodies and antigens. For example, a biomolecule may include a receptor or a capture antibody, a detection antibody, an enzyme, a substrate, a marker and/or an analyte, to name a few, or any combination or a complex formed by these molecules. It will be appreciated that the analyte may be an organic compound or an inorganic compound. When the analyte is an inorganic compound, a compound formed the analyte and another biomolecule, e.g., a capture antibody or a detection antibody, may be collectively referred to as a biomolecule.

As described above, the inventors have realized that, in accordance with embodiments disclosed herein, increasing active surface areas of detection structures of biosensor assemblies can increase the speed and sensitivity of immunoassay techniques. The active areas are configured for immobilizing biomolecules directly or indirectly thereon. Increasing the active area can increase the density of immobilized biomolecules, which can in turn increase the sensitivity of the immunoassay, e.g., ELISA. In addition, in accordance with embodiments of biosensor assemblies disclosed herein, the active surface area can be increased while reducing some of the possible negative impact on diffusive transport of various biomolecules, reagents and/or analytes. To address these and other needs, according to various embodiments, biosensor assemblies according to some embodiments have active surface areas that comprise a textured or modified surface, e.g., a textured or modified polymeric surface, which can have microstructures or nanostructures, according to various embodiments. As used herein, a microstructure has one or more physical dimensions, e.g., length, width, height, diameter, etc., that are about 100 nm to about 500 μm. A nanostructure has one or more physical dimensions that are less than about 100 nm.

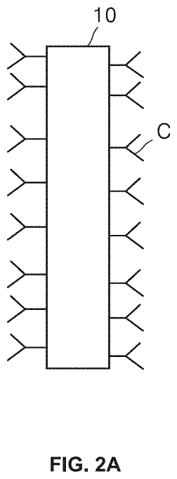

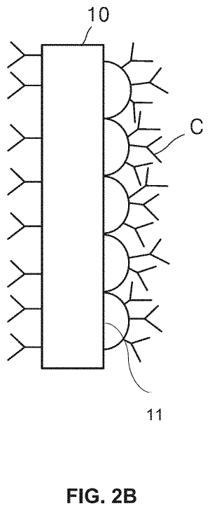

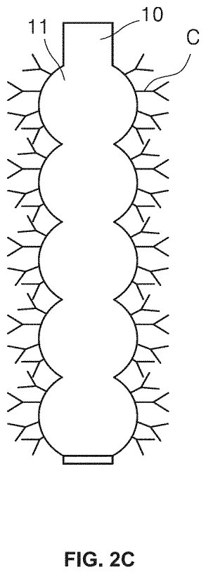

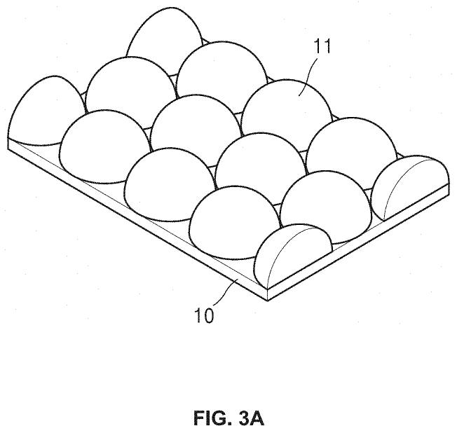

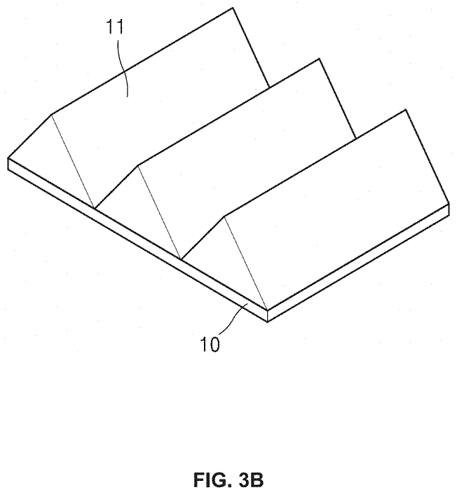

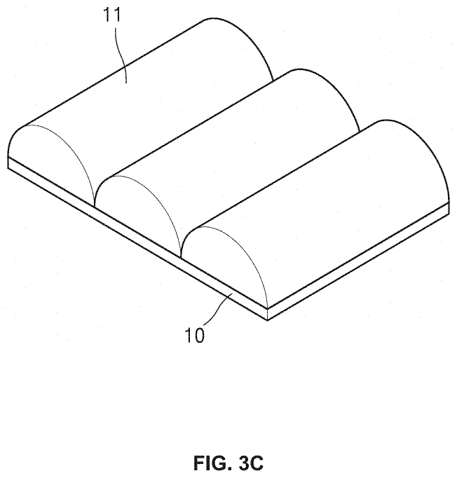

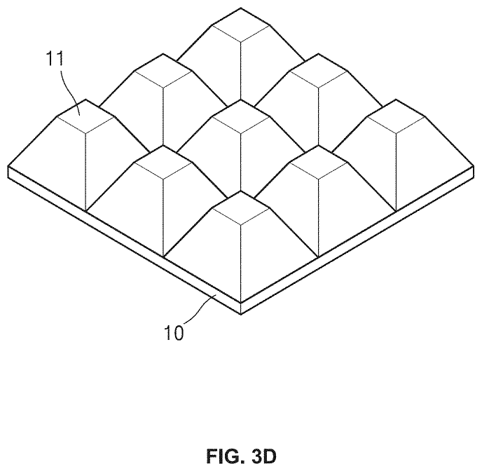

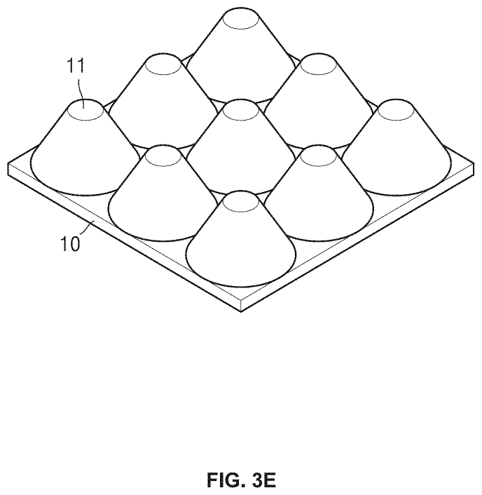

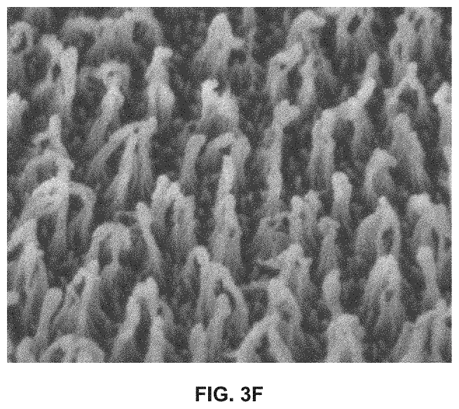

FIGS. 2A to 2C are cross-sectional or side views schematically illustrating detection structures 10 of biosensors having one or more major surfaces on which a biomolecule attached thereto. It will be appreciated that, while for illustrative purposes, the embodiments depict a receptor or a capture antibody C attached to the one or more major surfaces, embodiments are not so limited. In various embodiments, the biomolecule attached to the one or more major surfaces can be any biomolecule described herein that may be directly or indirectly attached to the one or more major surfaces. The biomolecule may be, e.g., an antibody such as a capture antibody, a target analyte and/or a detection antibody, according to various exemplary embodiments. The detection structures 10 illustrated in FIGS. 2B and 2C can also have one or more major surfaces that are textured or include microstructures or nanostructures 11. FIGS. 3A to 3E are perspective views schematically illustrating various examples of microstructures or nanostructures 11 formed on one or more major surfaces of biosensors according to some other exemplary embodiments.

FIG. 3F is a scanning electron microscope image of nanopillars or nanofibers formed on biosensors according to exemplary embodiments. The nanopillars or nanofibers were formed by etching in Ar and CF₄ plasma.

As illustrated in FIGS. 2A to 2C, a biosensor according to some embodiments includes a detection structure 10 in the shape of a plate having one or more major surfaces, e.g., a first surface and a second surface opposite the first surface. In the illustrated embodiments, each of the first and second major surfaces that oppose each other includes an active surface. However, embodiments are not so limited and in some other embodiments, one but not the other of the first and second major surfaces that oppose each other includes an active surface. In some embodiments, one or both of the first and second surfaces comprise an active surface configured to immobilize biomolecules directly or indirectly thereon. In some embodiments, e.g., embodiments illustrated with respect to FIGS. 2B and 2C, one or more major surfaces, e.g., the first and second major surfaces that oppose each other, can include a textured polymeric surface having microstructures or nanostructures 11. In some embodiments, the microstructures or nanostructures 11 can be formed of a polymeric material, e.g., the same or different polymeric material as the bulk of the detection structure 10.

As described herein, an active surface refers to a surface on which one or more biomolecules and/or analytes can be immobilized for an immunoassay. The active surface may be chemically treated or functionalized such that biomolecules and/or analytes, e.g., as antibodies or antigens, can be specifically bound, relative to non-active surfaces. For example, when exposed to the same solution under the same condition, an active surface may have a higher specificity to a particular antibody or an analyte by a factor exceeding, e.g., 2, 4, 6, 8, 10 or higher values compared to a non-active surface.

Referring to FIGS. 2A-2C, an immobilized biomolecule C or a reagent, e.g., an antibody, is arranged on at least one of the main surfaces, e.g., flat first and second surfaces, of a planar detection structure 10. Here, for illustrative purposes, the immobilized biomolecule C is a biomolecule, e.g., a capture antibody, that is configured to be specifically bound or is specifically bound to a target analyte. The target analyte may be independently provided or may be present in a sample. The sample may further include a marker-labeled detection biomolecule or a reagent, e.g., an antibody.

A target analyte may be an organic compound or an inorganic compound. Non-limiting examples of target analytes include: amino acids, peptides, polypeptides, proteins, glycoproteins, lipoproteins, nucleosides, nucleotides, oligonucleotides, nucleic acids, sugars, carbohydrates, oligosaccharides, polysaccharides, fatty acids, lipid, hormones, metabolites, cytokines, chemokines, receptors, neurotransmitters, antigens, allergens, antibodies, substrates, cofactors, inhibitors, drugs, pharmaceuticals, nutrients, prions, toxins, poisons, explosives, pesticides, chemical warfare agents, biohazardous agents, bacteria, viruses, radioisotopes, vitamins, heterocyclic aromatic compounds, carcinogens, mutagens, narcotics, amphetamines, barbiturates, hallucinogens, waste products, contaminants, and mixtures thereof.

It will be appreciated that the immobilized biomolecule C or a reagent, e.g., an antibody such as a capture antibody or a detection antibody, that is configured to specifically bind or is bound to the target analyte is determined depending on the target analyte. Non-limiting examples of the immobilized biomolecule C include: low molecular weight compounds, antigens, antibodies, proteins, peptides, deoxyribonucleic acid (DNA), ribonucleic acid (RNA), peptide nucleic acid (PNA), enzymes, enzyme substrates, hormones, hormone receptors, and synthetic reagents having functional substrates, mimics thereof, and combinations thereof.

Non-limiting examples of a marker for labeling an antibody include: horseradish peroxidase (HRP), alkaline phosphatases, and fluoresceins, to name a few.

Non-limiting examples of a substrate solution include: 2,2′-azino-bis[3-ethylbenzothiazoline-6-sulfonic acid] diammonium salt (ABTS) or 3,3′,5,5′-tetramethylbenzidine (TMB) as a reagent reacting with the marker, to name a few. In some embodiments, any enzymatic substrate can be employed in the methods, kits and devices provided herein. In some embodiments, the substrate is at least one or more of: OPD (o-phenylenediamine dihydrochloride), and/or pNPP (p-nitrophenyl phosphate, disodium salt).

It will be appreciated that the disclosed examples of the target analyte, the immobilized biomolecule C, the marker, and the reagent are provided as non-limiting examples and are not necessarily limited thereto.

FIGS. 3A to 3E are perspective views schematically illustrating various examples of microstructures or nanostructures 11 formed on one or more major surfaces of detection structures 10 of biosensors according to exemplary embodiments. As illustrated in FIGS. 3A to 3E, microstructures or nanostructures 11 may be formed on at least one of major surfaces, e.g., first and/or second surfaces of the detection structure 10. In some embodiments, the first and second surfaces may be opposing major surfaces. However, embodiments are not so limited and in some other embodiments, the first and second surfaces may be adjoining surfaces. The microstructures or nanostructures 11 may be formed over the entire area or substantially the entire area of the first surface and/or the second surface of the detection structure 10. Alternatively, the microstructures or nanostructures 11 may be formed in some sections or portions of the first surface and/or the second surface of the detection structure 10. The immobilized biomolecule C may be arranged on the outer surface of at least some or substantially all of the microstructures or nanostructures 11. With this arrangement, a relatively larger amount of the immobilized biomolecule C can be formed on a surface of the detection structure 10 compared to a detection structure that does not have the microstructures or nanostructures 11 formed thereon, because the microstructures or nanostructures 11 provide a relatively larger surface area than a planar detection structure that does not have the microstructures or nanostructures 11 formed thereon (e.g., FIG. 2A).

The microstructures or nanostructures 11 may take the form of protrusions or projections. In various embodiments, the microstructures or nanostructures 11 comprise protrusions having cross sectional areas that decrease away from a base, i.e., closer to the solid substrate on which they formed. Advantageously, these structures can significantly enhance the surface area available for immobilization of the biomolecules C and/or analytes while reducing possible negative impact on diffusive transport of various reagents and/or analytes. The microstructures or nanostructures 11 can have any suitable shape that can increase an active surface area for immobilization of biomolecules thereon, as described herein. The suitable shape can have at least one surface that at least partly approximate a polygon, a circle or an oval. For example, the microstructure or nanostructure 11 can have a shape that includes at least a portion of a sphere, an ovoid, a pyramid (e.g. rectangular, triangular), a prism (e.g., rectangular, triangular), a cone, a cube, a cylinder, a plate, a disc, a wire, a rod, a sheet and fractals, to name a few. The microstructures or nanostructures 11 can include any truncated portions or distorted forms of these various shapes.

In some embodiments, as illustrated in FIG. 3A, the microstructures or nanostructures 11 include projections that have the shape of a truncated sphere, e.g., a hemispherical shape. The hemispherical projections may be arranged regularly, e.g., as a regular array. In the illustrated embodiment, the projections are arranged in a zigzag configuration. This configuration allows the adjacent projections to be in contact with one another in a two-dimensionally close-packed configuration. However, embodiments are not so limited and in some other embodiments, the projections may be arranged in any suitable configuration, e.g., in a rectangular array or randomly. As used herein, a microstructure has one or more physical dimensions, e.g., length, width, height, diameter, etc., that are about 100 nm to about 500 μm. A nanostructure has one or more physical dimensions that are less than about 100 nm.

In other embodiments, the projections may be prismatic (see FIG. 3B) or semi-cylindrical (see FIG. 3C).

In yet other embodiments the projections may be pyramidal or conical. For example, the projections may have the shape of a pyramidal frustrum (see FIG. 3D) or a conical frustrum (see FIG. 3E) having cross-sectional areas that are tapered in the direction perpendicular to a major surface of the detection structure 10.

The use of the microstructures or nanostructures 11 with large surface area can increase the amount and/or concentration of a receptor or antibody participating in a reaction, achieving improved sensitivity compared to conventional ELISA techniques. Particularly, the use of the microstructures or nanostructures 11 can effectively control the hook effect caused when a protein is present at a higher concentration than a receptor or antibody.

In various embodiments, the microstructure or nanostructure 11 has an average value of a lateral base dimension (e.g., a hemisphere diameter in FIG. 3A, a prism width in FIG. 3B, a hemicylinder width in FIG. 3C, a pyramidal frustrum base width in FIG. 3D, a conical frustrum base diameter in FIG. 3E, or a fiber diameter in FIG. 3F) that is about 1-10 nm, 10-100 nm, 100-1000 nm, 0.1-1 μm, 1-10 mm, 10-100 μm, 100-500 μm, or a value in a range defined by any of these values.

In some embodiments, the microstructures or nanostructures 10 are regularly arranged, e.g., periodically arranged. However, embodiments are not so limited and in other embodiments, the microstructures or nanostructures can be randomly arranged or pseudo-randomly arranged, e.g., regularly arranged in one direction while randomly arranged in another direction.

### Cuvette-Type Sensor Assemblies Configured for Enhanced Sensitivity and Reagent Diffusion

The biosensors according to some embodiments may be of a cuvette-type or strip-type, which will be separately explained in detail. As described above, it is advantageous to have biosensor assemblies having increased active surface areas of detection structures while also reducing possible negative impact on diffusive transport of various biomolecules, reagents and/or analytes. To address these and other needs, according to various embodiments, biosensor assemblies according to various embodiments have a partly or entirely transparent container having one or more cavities formed therein. The one or more cavities are configured to hold a liquid sample. The container can include a plurality of active surfaces disposed in each of the one or more cavities that are configured for immobilizing a biomolecule or a reagent thereon. The biosensors additionally include one or more detection structures, which can be partly or entirely transparent or opaque, that are configured to be at least partially disposed in each of the one or more cavities, such that when the one or move cavities are filled with a liquid sample, the one or more transparent detection structures are configured to be at least partially immersed in the liquid sample. Each of the detection structures comprises one or more main surfaces that provide one or more of the active surfaces. As configured, the plurality of active surfaces increases the available active surface area for immobilizing biomolecules and/or analytes thereon. In addition, each of the active surfaces is separated from an immediately adjacent one of the active surfaces by a suitable distance to facilitate diffusive transport or reduce retardation of diffusive transport of various biomolecules, reagents and/or analytes involved in ELISA processes.

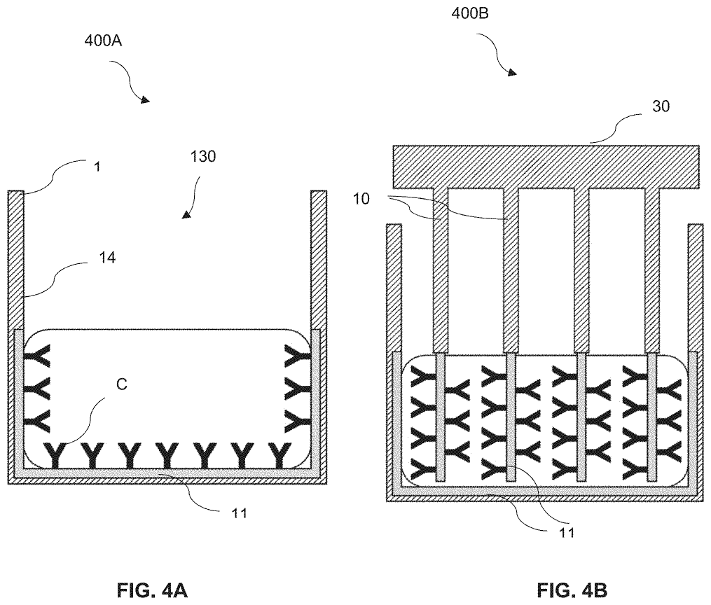

FIG. 4A is a schematic cross-sectional view of a cuvette-type biosensor assembly 400A, according to some embodiments. The biosensor assembly illustrated in FIG. 4A includes a cuvette 1 having a cavity 130 formed therein for holding a liquid sample. An active area 11 covers at least a part of the inner surfaces of the cavity 130 of the cuvette 1. As described herein, the active area 11 is configured to immobilize a biomolecule C at a higher specificity compared to an inactive area 14.

FIG. 4B is a schematic cross-sectional view of a cuvette-type biosensor assembly 400B, according to some other embodiments. The biosensor assembly 400B illustrated in FIG. 4B includes a cuvette 1 that can be similarly arranged as in the illustrated embodiment of FIG. 4A, including the cavity 130, which may or may not have the active area 11 formed on the sidewalls. Unlike the cuvette-type biosensor described above with respect to FIG. 4A, in addition to having an active area 11 covering at least a part of the inner surfaces of the cavity 130 of the cuvette 1, the biosensor assembly 400B additionally includes one or more transparent detection structures 10 disposed in the cavity 130, wherein each of the transparent detection structures 11 comprises one or more main surfaces that provide one or more of the active surfaces 11. As configured, the plurality of active surfaces increases the available active surface area for immobilizing biomolecules and/or analytes thereon. In addition, each of the active surfaces 11 is separated from an immediately adjacent one of the active surfaces 11 or an inactive surface by a suitable distance to facilitate or reduce retardation of diffusive transport of various biomolecules, reagents and/or analytes involved in ELISA processes.

The inventors have discovered that the suitable distance or gap between immediately adjacent surfaces for unhindered diffusive transport of various biomolecules, reagents and/or analytes used in an ELISA is about 500 microns, 1000 microns, 2000 microns, 3000 microns, 4000 microns, 5000 microns or 6000 microns, 7000 microns, 8000 microns, 9000 microns or a distance in a range defined by any of these values, depending on the particular configuration of the biosensor assembly and the target analyte to be detected or quantified. For a given configuration of the biosensor assembly, in order to achieve the various advantageous experimental results described herein including high sensitivity and fast reaction times, the inventors have discovered that it can be critical to have a separation distance between immediately adjacent surfaces, wherein at least one of the surfaces is an active surface, that is greater than or equal to one or more of these values.

The inventors have also discovered that a suitable combined active area for immobilization of various biomolecules and/or analytes used in an ELISA per volume of the cavity exceeds about 0.1 mm²/μl, 1.0 mm²/μl, 1.5 mm²/μl, 2.0 mm²/μl, 2.5 mm²/μl, 3.0 mm²/μl, 3.5 mm²/μl, 4.0 mm²/μl, 4.5 mm²/μl, 5.0 mm²/μl 5.5 mm²/μl, 6.0 mm²/μl, 6.5 mm²/μl, 7.0 mm²/μl, 7.5 mm²/μl, 8.0 mm²/μl, or has a value in a range defined by any of these values, depending on the particular configuration of the biosensor assembly and the target analyte to be detected or quantified. For a given configuration of the biosensor assembly, in order to achieve the various advantageous experimental results described herein including high sensitivity and fast reaction times, the inventors have discovered that it can be critical to have a combined active surface area that is greater than or equal to one or more of these values.

The inventors have also discovered that, when the active areas of the sensor assemblies are configured as described herein, a detectable concentration of the analyte specifically bound to the immobilized biomolecule or reagent, and/or a detectable optical density resulting therefrom, is increased by at least 1.1, 2, 5, 10, 15, 20 times, or by a factor in a range defined by any of these values, without substantially decreasing a rate of specifically binding the analyte, e.g., antigens, to the biomolecules, e.g., antibodies, relative to comparable sensor assemblies without the one or more transparent detection structures. For example, the sensor assemblies 400B and 400A with and without detection structures 10 illustrated in FIG. 4B and FIG. 4A, respectively, can have these ratios of specifically bound analytes.

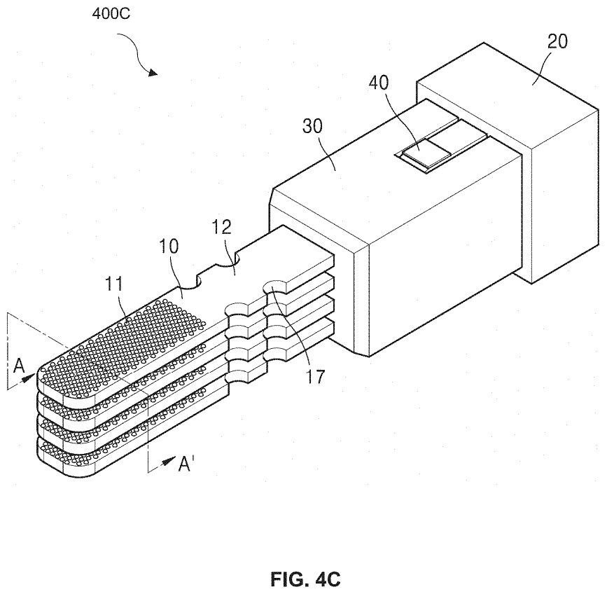

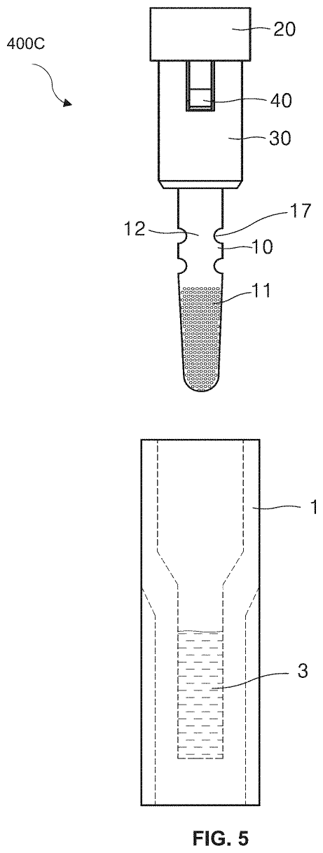

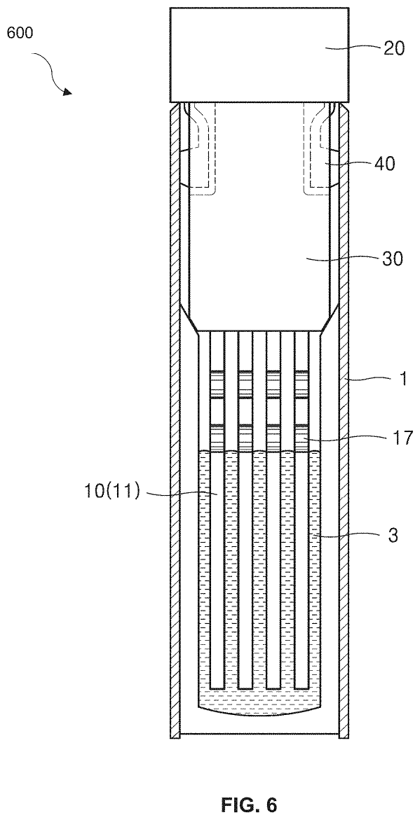

FIG. 4C is a perspective view of a cuvette-type biosensor 400C according to one embodiment of the disclosed technology. FIG. 5 illustrates front views of the biosensor 400C illustrated in FIG. 4C and a cuvette 1 into which the biosensor 400C is configured to be inserted, and FIG. 6 is a side view 600 illustrating a state in which the biosensor 400C illustrated in FIG. 5 is inserted into the cuvette 1.

As illustrated in FIGS. 4B to 6, the cuvette-type biosensor assembly according to various embodiments has a structure in which the detection structures 10 are inserted into the cuvette 1. Due to this structure, an immobilized biomolecule arranged on the detection structures 10 reacts with a target analyte accommodated in the cuvette 1. The target analyte may be present in a sample 3 accommodated in the cuvette 1. In this case, the immobilized biomolecule reacts with the target analyte while the detection structures 10 are immersed in the sample 3. As described above, the immobilized biomolecule is arranged on the detection structures 10. In some embodiments, the detection structures 10 may have formed thereon microstructures or nanostructures 11 as described above, e.g., with respect to FIGS. 2B-3E. The microstructures or nanostructures 11 may be formed over at least a portion of an active area of each of the main surfaces of the detection structures 10. In some embodiments, the entire areas of a first surface and/or a second surface, which may be opposing major surfaces of each of the detection structures 10, are configured as active areas, such that they are covered with the microstructures or nanostructures 11. Alternatively, portions of the first and/or second surfaces of the detection structures 10 are configured as active areas, such that the microstructures or nano structures 11 are formed selectively on some portions of the first surface and/or the second surface but not on other portions. The immobilized biomolecule may be arranged on the outer surfaces of the detection structures 10. Absorbance is measured in a state in which the detection structures 10 are inserted into the cuvette 1. Accordingly, the detection structures 10 and the cuvette 1 may have predetermined absorbances, e.g., to provide reference absorbance curves.

Advantageously, one or more of the detection structures 10 and the cuvette 1 as illustrated in FIGS. 4A-6 may be formed of a solid polymeric material. For example, the detection structures 10 and the cuvette 1 may be made of polymer materials such as polycarbonate, polyethylene terephthalate, polymethyl methacrylate, triacetyl cellulose, cycloolefin, polyarylate, polyacrylate, polyethylene naphthalate, polybutylene terephthalate or polyamide. However, these polymer materials are merely illustrative and other suitable light-transmitting polymer materials may be used without particular limitation. It will be appreciated that, in addition to providing optical transparency for immunoassays, the selection of a suitable material for the detection structure 10 and/or the cuvette 1 can advantageously enable effective formation of the active surfaces by surface chemical modification or functionalization. In addition, the selection of a suitable material for the detection structure 10 and/or the cuvette 1 can advantageously enable formation of a suitable surface morphology, including microstructures or nanostructures 11 (FIGS. 3A-3E) by direct modification of the surface thereof. For example, any of the microstructures or nanostructures 11 described herein for enhancement of the surface area can be formed as an integral part of a solid substrate, e.g., a solid polymeric substrate. The microstructures or nanostructures 11 can be formed directly on surfaces of the detection structure 10 and/or the cuvette 1 by a suitable process. For example, the microstructures or nanostructures 11 can be molded as part of the solid polymer substrate, or can be formed by a suitable post-processing method, e.g., by etching or plasma processing. Thus, in some embodiments, each of the active surfaces comprises a solid polymeric surface that serves directly as the active surface for immobilizing biomolecules and/or analytes, without forming a separate layer with topography. In these embodiments, the active surfaces may not include an additional material formed thereon to serve as active surfaces. That is, the surface on which biomolecules are immobilized may be the same material as solid substrate. When the solid substrate is formed of a polymeric material, the active surface may not have another material, e.g., a metal, a semiconductor, an inorganic glass or a polymeric material different from the polymeric material of the solid substrate may not be present between the solid substrate and the biomolecules attached thereto. However, embodiments are no so limited and in other embodiments, the active surfaces may have formed thereon an additional material.

Referring to FIGS. 4C-6, the cuvette-type biosensor may further include a gripping member or a handle 20 connected to one end of each of the detection structures 10. The gripping member 20 forms a base from which the detection structures 10 extend and is configured to be gripped by a user. The user can insert the detection structures 10 into the cuvette 1 while holding the gripping member 20. At this time, the free ends of the detection structures 10 first enter the cuvette 1. The inserted detection structures 10 are immersed in the sample 3. The free ends of the detection structures 10 refer to the opposite ends to the ends of the detection structures 10 connected to the gripping member 20.

Each of the detection structures 10 is fixedly connected to the gripping member 20. The detection structures 10 are spaced apart from and parallel to each other such that major surfaces of adjacent detection structures 10 face each other. The separation distance between immediately adjacent active surfaces can be any one of distances described herein, and can be critical to achieve the various advantages associated with increased optical density and/or reduced reaction time. The formation of the plurality of detection structures 10 increases the density (or concentration) of the immobilized biomolecule per unit volume of the sample 3 containing the analyte by increasing the surface area available for the immobilization of the biomolecules. As a result, improved sensitivity of the sensor and control over the hook effect can be achieved.

Still referring to FIGS. 4C-6, the cuvette-type biosensor may further include a cap 30. The cap 30 is configured to be releasably inserted into the open inlet or the opening of the cuvette 1 to close the inlet of the open cuvette 1. The inlet of the cuvette 1 can be substantially or completely sealed or closed by the cap 30. Alternatively, only a portion of the inlet of the cuvette 1 may be closed by the cap 30. The cap 30 is disposed under the gripping member 20 to connect the gripping member 20 to the detection structures 10. The cap 30 is held in contact with the inner surface of the cuvette 1 to prevent the movement of the detection structures 10 in the cuvette 1.

The inventors have realized that, depending on the size of the inlet of the cuvette 1, there may be a clearance between the outer surface of the cap 30 and the inner surface of the inlet of the cuvette 1. Thus, the cap 30 may remain unfixed to the cuvette 1, making it difficult to accurately analyze the sample 3 without, e.g., leaking the sample 3. To avoid this problem, the biosensor 400C (FIGS. 4C, 5, 6) may further include a fixing member or a securing member 40 for fixing or securing the detection structures 10 irrespective of a close match between relative sizes of the inlet of the cuvette 1 and the cap 30.

The fixing member 40 is arranged on the outer surface of the cap 30. With this arrangement, the original location or shape of the fixing member 40 is changed to create resilience when the cap 30 is inserted into the cuvette 1. The fixing member 40 is brought into close contact with the inner circumferential surface of the cuvette 1 by the resilience.

For example, referring to FIGS. 5 and 6, when the cap 30 is inserted into the cuvette 1, the fixing member 40 may be pressurized and deformed, e.g., elastically bent or deformed, by the inner surface of the cuvette 1. Accordingly, the fixing member 40 may be made of an elastic material such as a rubber. The elasticity of the elastic material allows the fixing member 40 to come into contact with the inner surface of the inlet of the cuvette 1. The fixing member 40 may fixedly hold the cap 30 and the detection structures 10 in place using the inherent elasticity of the elastic material. Alternatively, the fixing member 40 may include an elastic member, such as a spring. In this case, the fixing member 40 may fixedly hold the cap 30 and the detection structures 10 in place using the elastic force of the spring. However, embodiments are no so limited and the fixing member 40 may not necessarily be dependent on the elasticity of the elastic material or member. For example, the fixing member 40 may be structurally modified such that the detection structures 10 are fixed, which will be explained in detail below.

In some embodiments, the fixing member 40 may extend from the outer surface of the cap 30 and may be bent in a predetermined direction. For example, the fixing member 40 may extend outward from the outer surface of the cap 30 and may be bent in parallel to the outer surface of the cap 30 to form an inverted L shape. The outwardly protruding protrusion formed at one end of the fixing member 40 is pressurized against the inner surface of the cuvette 1, and as a result, the fixing member 40 can be brought into close contact with the inner surface of the cuvette 1 by tension. At this time, since the fixing member 40 is moved toward the cap 30 when pressured, a portion of the outer surface of the cap 30 opposite to the fixing member 40 may be recessed.

Alternatively, the fixing member 40 may extend to the recessed portion of the cap 30 and may protrude outward from the outer surface of the cap 30 to form an “L” shape.

In conclusion, the fixing member 40 may be freely modified so long as it can be brought into close contact with the inner surface of the cuvette 1 when the cap 30 is inserted into the cuvette 1.

Referring to FIGS. 5 and 6, when immersed in the sample 3, each of the detection structures 10 may be divided into an immersion portion immersed in the sample 3 and a non-immersion portion above the liquid sample including a narrow portion 12 whose width is smaller than that of the immersion portion.

When the detection structures 10 are inserted into the cuvette 1 to analyze the sample 3, a capillary force is created in the gaps between the one or both of the outer ones of the detection structures 10 and the respective inner surface(s) of the cuvette 1, and/or in the gap(s) between the detection structures 10 arranged in parallel. The capillary force may increase the level of the sample 3, requiring a larger amount of the sample 3 for analysis, which can and seriously deteriorate the analytical reliability of the sensor. The inventors have realized that such problems may be mitigated or solved by the formation of the narrow portions 12. The level of the sample 3 rises along the detection structures 10 by an attractive force between the sample 3 and the surfaces of the detection structures 10. Accordingly, the formation of the narrow portions 12 with a smaller width reduces the contact areas between the detection structures 10 and the sample 3 to reduce or prevent the level of the sample 3 from rising.

Still referring to FIGS. 5 and 6, the narrow portions 12 may have one or more recesses, notches or indentations 17 depressed from one or both sides of the detection structures 10. The anti-rising recesses 17 are formed to a predetermined depth in the direction from one side to the other opposing side of the detection structure 10. Accordingly, the width of the detection structure 10, i.e., the distance between opposing sides of the detection structure 10, at the position where the anti-rising recesses 17 are formed is reduced to be smaller than the width of the detection structure at the immersion portion below the lowest recesses 17. The anti-rising recesses 17 may be formed at one or both sides of the detection structure 10. When formed on both sides, the anti-rising recesses 17 at both sides of the detection structure 10 may be arranged at the same vertical level. However, embodiments are not necessarily limited to this arrangement. For example, the anti-rising recesses 17 may be alternately arranged in a zigzag pattern. The anti-rising recesses 17 may be formed at predetermined intervals in the lengthwise direction along the sides of the detection structure 10.

The anti-rising recesses 17 may be rounded in shape as illustrated, but are not necessarily limited to this shape. The anti-rising recesses 17 may have any shape so long as the width of the detection structures 10 is narrowed.

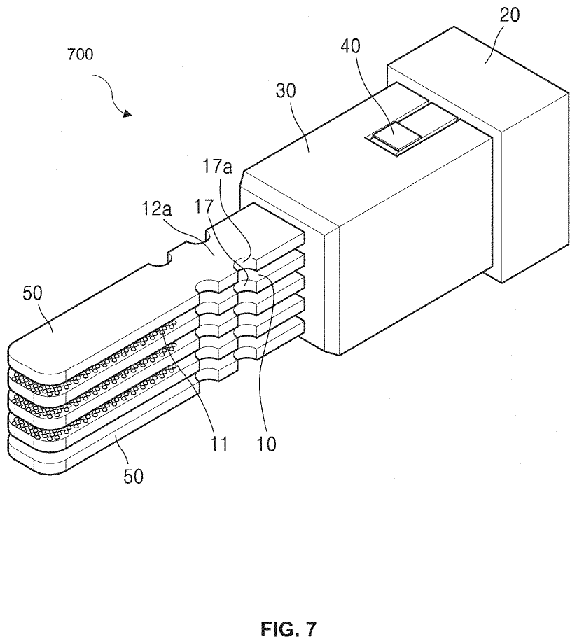

FIG. 7 is a perspective view of a cuvette-type biosensor 700 according to a further embodiment of the disclosed technology. The biosensor 700 has various features that are similar to the biosensors illustrated above with respect to FIGS. 4C-6, a detailed description of which are not repeated herein for brevity. In the illustrated embodiment in FIG. 7, the biosensor 700 further includes one or more guards 50 configured to protect detection structures 10. In the illustrated embodiment, a pair of guards 50 face each other through and are interposed by the detection structures 10 and are spaced apart from the detection structures 10. The number of the detection structures 10 interposed between the pair of guards 50 is not limited to any particular number, but may be limited by the inner dimensions of the cuvette 1, the thicknesses of the detection structures 10 and the spacings between adjacent detection structures 10. By being configured as outermost structures, the guards 50 prevent the detection structures 10 from being in contact with the inner surfaces of the cuvette 1 and protect the detection structures 10 from external factors such as impacts. The guards 50 may be in the shape of plates as illustrated, but are not necessarily limited to this shape. When the guards 50 are provided in the shape of plates, the sample 3 may rise in the gaps between the detection structures 10 arranged in parallel or in the gaps between the detection structures 10 and the inner surface of the cuvette 1. Thus, in a similar manner as the detection structures described above with respect to FIGS. 5 and 6, the guards 50 may include narrow portions 12a whose width is smaller than portions that are immersed, formed at a predetermined height in the guards 50. The narrow portions 12a may have recesses 17a depressed from the sides of the guards 50. However, the formation of the narrow portions 12a in the guards 50 or the formation of the anti-rising recesses 17a in the narrow portions 12a are not necessarily required.

In some embodiments, neither of opposing major surfaces of one or both of the guards 50 may be configured for immobilization of biomolecules and/or have microstructures or nanostructures formed thereon. In some embodiments, one of the opposing major surfaces of one or both of the guards 50, e.g., the major surface facing the detection structures 10, may be configured for immobilization of biomolecules and/or have microstructures or nanostructures formed thereon. In yet some embodiments, an immobilized biomolecule may be attached to one or both surfaces of one or both of the guards 50. As a result, the density of the immobilized biomolecule per unit volume can be increased.

In each of the embodiments described herein, e.g., with respect to FIGS. 2A to 7, the detection structures 10 may have a thickness about 100 to 5000 microns, 100 to 500 microns, 500 to 1000 microns, 1000 to 1500 microns, 1500 to 2000 microns, 2000 to 2500 microns, 2500 to 3000 microns, 3000 to 3500 microns, 3500 to 4000 microns, 4000 microns to 4500 microns, 4500 to 5000 microns, or a thickness in a range defined by any of these values.

In each of the embodiments described herein, e.g., with respect to FIGS. 2A to 7, the detection structures 10 may have one or more main surfaces, e.g., first and second surfaces, each having an area of about 10 to 100 mm², 10 to 20 mm², 20 to 30 mm², 30 to 40 mm², 40 to 50 mm², 50 to 60 mm², 60 to 70 mm², 70 to 80 mm², 80 to 90 mm², 90 to 100 mm², or a surface area in a range defined by any of these values, for instance about 51 mm². Furthermore, in each of the embodiments described herein, e.g., with respect to FIGS. 2A to 7, a suitable portion of each of the main surfaces, e.g., 30-40%, 40-50%, 50-60%, 60-70%, 70-80%, 80-90%, 90-100%, or any percentage in a range defined by these values, may be active surfaces.

In each of the embodiments described herein, e.g., with respect to FIGS. 2A to 7, the cuvette 1 is configured to hold a liquid having a volume of about 50 mm³-3000 mm³, 50 mm³-300 mm³, 300 mm³-600 mm³, 600 mm³-900 mm³, 900 mm³-1200 mm³, 1200 mm³-1500 mm³, 1500 mm³-1800 mm³, 1800 mm³-2100 mm³, 2100 mm³-2400 mm³, 2400 mm³-2700 mm³, 2700 mm³-3000 mm³, or a value in a range defined by any of these values.

In each of the embodiments described herein, e.g., with respect to FIGS. 2A to 7, the cuvette 1 and the detection structures 10 have suitable dimensions, including suitable separation distances between adjacent surfaces, e.g., between active surface, and a suitable combined active area for immobilization of various biomolecules and/or analytes, such that the cuvette 1 is configured to receive 1 to 20, 1 to 5, 5 to 10, 10 to 15, 15 to 20 or any number of detection structures in a range defined by any of these values.

### Strip-Type Sensor Assemblies Configured for Enhanced Sensitivity and Reagent Diffusion

Hereinafter, strip-type biosensor assemblies according to embodiments are described. In these embodiments, an optically transparent container configured to receive one or more detection structures comprises a strip container comprising a plurality of cavities formed therein. Unlike the cuvette-type biosensor assemblies described above, in which one or more detection structures may have a plate structure having opposing main surfaces that may be substantially parallel to a depth direction of the cavities, in the strip-type biosensors disclosed herein, the one or more transparent detection structures comprises a plate structure having opposing main surfaces that are substantially perpendicular to a depth direction the cavities. In some embodiments, each of the one or more transparent detection structures comprises a plate structure having opposing main surfaces that are substantially parallel to each other. A main surface of one of the one or more transparent detection structures directly facing a bottom surface of a respective one of the cavities may be substantially parallel to the bottom surface of the respective one of the cavities, and may be separated from the bottom surface of the respective one of the cavities by the distance exceeding about 500 microns.

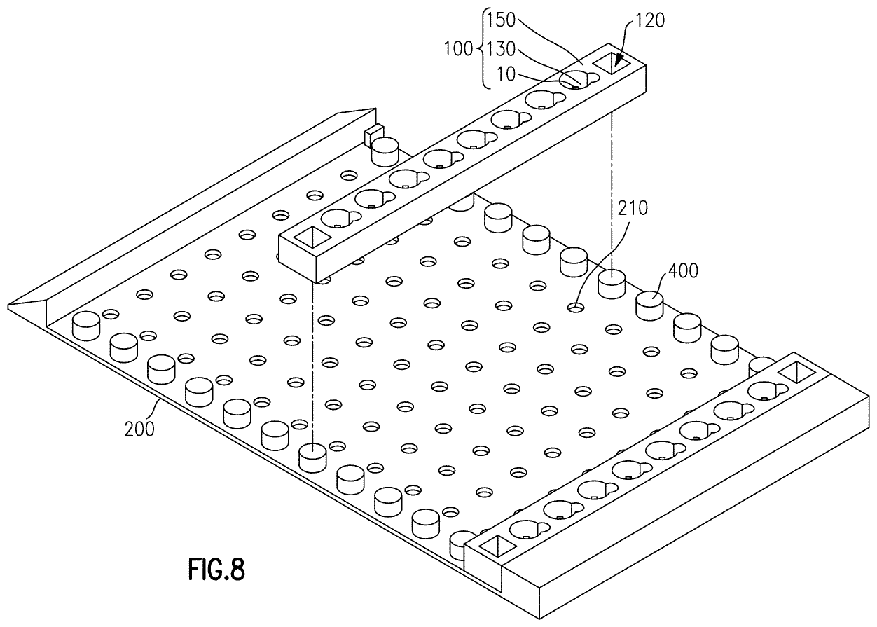

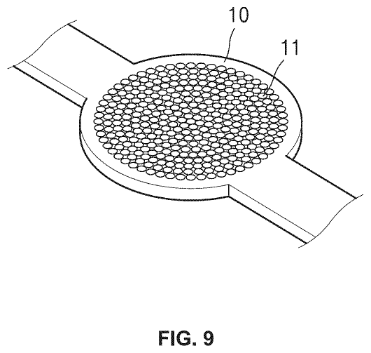

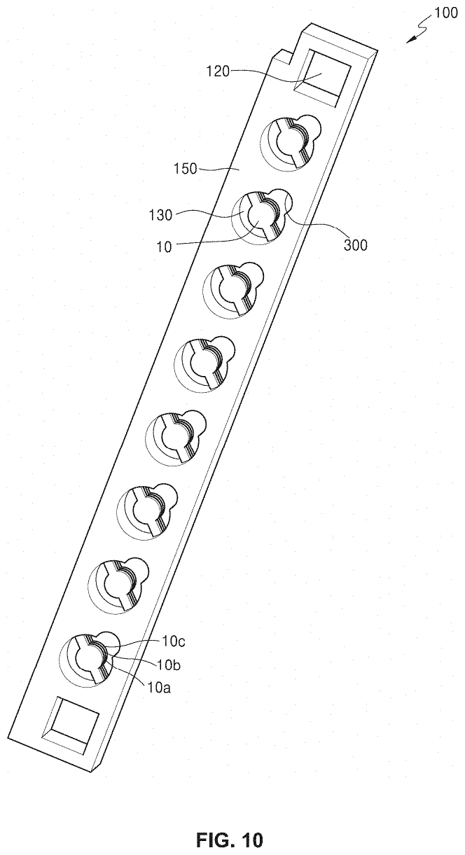

In some embodiments, each of the one or more transparent detection structures comprises a plate structure disposed laterally at a central region of a respective one of the cavities, as exemplified by various embodiments described herein including those with respect to FIGS. 8-11. In some embodiments, one or more of the main surfaces of the one or more transparent detection structures may substantially overlap one another in a vertical direction substantially parallel to a depth direction of the cavities. In some embodiments, the sensor assembly comprises two or more transparent detection structures, wherein main surfaces of directly adjacent ones of the two or more transparent detection structures directly facing each other are separated from each other by the distance exceeding 500 microns. FIG. 8 is a perspective view of a strip-type biosensor according to one embodiment of the disclosed technology, FIG. 9 is a perspective view schematically illustrating a detection structure of a strip-type biosensor according to some embodiments, and FIG. 10 is a perspective view illustrating a sensor strip of a strip-type biosensor according to some embodiments.

As illustrated in FIGS. 8 to 10, the strip-type biosensor assembly according to embodiments includes one or more sensor strips 100, each of which includes a body 150 with a predetermined length and a plurality of reaction chambers 130, wells or cavities, recessed from one surface of the body 150 to accommodate a target analyte-containing sample. One or more detection structures 10 are arranged in each of the reaction chambers 130.

Specifically, the strip-type biosensor assemblies according to some embodiments includes sensor strips 100, each of which has a structure in which reaction chambers 130 are formed in a body 150 in the shape of an elongated strip with a predetermined length and width. The body 150 includes one or more chambers 130 in the form of a recessed well or cavity, and at least one detection structure 10 is arranged in each of the reaction chambers 130.

The reaction chambers 130 are recessed from one of the outer surfaces of the body 150 to accommodate a sample therein and are arranged along the lengthwise direction of the body 150. An immobilized biomolecule may be arranged at least on bottom surfaces (not shown) of the reaction chambers 130. In some embodiments, microstructures or nanostructures 11 in the form of projections similar to the microstructures or nanostructures 11 described in the previous embodiments may be formed at the bottom surfaces of the reaction chambers 130, and the immobilized biomolecule is arranged on the outer surface of the microstructures or nanostructures in the form of projections. The formation of the microstructures or nanostructures 11 enhances the density of the immobilized biomolecule per unit volume, in a similar manner as described above. However, embodiments are not so limited and in some other embodiments, the microstructures or nanostructures 11 may be omitted from the bottom surfaces of the reaction chambers 130.

The detection structure 10 is arranged in each of the plurality of reaction chambers 130 such that it is configured to be immersed in the sample accommodated in the reaction chamber 130. In some embodiments, the detection structure 10 has a plate structure having opposing major planar surfaces. Referring to FIG. 9, in some embodiments, one or both of opposing planar surfaces have formed thereon microstructures or nanostructures 11, in a similar manner as described above with respect to cuvette-type sensor assemblies. In some embodiments, the microstructures or nanostructures 11 are formed over substantially the entire area of a first surface and/or a second surface of the detection structures 10 opposing the first surface. Alternatively, microstructures or nanostructures 11 may be formed in some but not other sections of the first surface and/or the second surface. The immobilized biomolecule may be arranged on the outer surfaces of the detection structure 10. However, embodiments are not so limited, and in some other embodiments, the microstructures or nanostructures 11 may be omitted from the surfaces of the detection structures 10.

The formation of the plurality of reaction chambers 130 and the detection structures 10 in each of the sensor strips 100 enables parallel or simultaneous analysis of a plurality of reactions. In some analysis techniques, different reactions may be analyzed using the same sample. For example, when different capture antibodies are immobilized on the detection structures 10 in different reaction chambers 130, different reactions may be analyzed using the same sample. In some other analysis techniques, different reactions may be analyzed using the different samples. For example, different reactions may be analyzed using different samples introduced into different reaction chambers. Still referring to FIG. 9, the opposite surface to the surface of the sensor strip 100 where the openings of the reaction chambers 130 are formed may be arranged on a fixing plate 200. The fixing plate 200 may be configured to fix a plurality of sensor strips 100. As arranged, n×m reactions may be analyzed, where n may represent the number of reaction chambers per sensor strip 100 and m may represent the number of sensor strips 100 that may be fixed on the fixing plate 200.

The fixing plate 200 has a predetermined width and thickness. The sensor strips 100 are detachably attached to one surface of the fixing plate 200. The sensor strips 100 can be attached to and detached from the fixing plate 200 by insertion protrusions 400 and insertion holes 120. The insertion protrusions 400 are inserted into and fixed to the insertion holes 120. The insertion holes 120 may be recessed or perforated so as to have a shape corresponding to the outer shape of the insertion protrusions 400. Due to their corresponding shapes, the insertion protrusions 400 are releasably withdrawn from the insertion holes 120. The insertion protrusions 400 may protrude from one surface of the fixing plate 200 and the insertion holes 120 may be formed on the opposite surface of the body 150 of the sensor strip 100 so that the sensor strip 100 can be attached to and detached from the fixing plate 200. Alternatively, the insertion protrusions 400 may be formed on the sensor strip 100 and the insertion holes 120 may be formed in the fixing plate 200.

The biosensor assemblies according to various embodiments are configured for analysis of a target analyte based on an absorbance measurement. Thus, the detection structures 10 are configured to be irradiated with external light. Accordingly, the fixing plate 200 may be perforated along its thickness direction to form holes 210 to pass unhindered light therethrough. The reaction chambers 130 are configured to be arranged over the holes 210 formed in the fixing plate 200. With this arrangement, the reaction chambers 130 and the holes 210 may be provided in the same number at their corresponding positions. However, embodiments are not so limited and in some other embodiments, the fixing plate 200 may comprise a frame configured to fix or support thereon one or more edges of the one or more sensor strips 100, while remaining portions are removed. For example, in some configurations, the plurality of holes 210 corresponding to a sensor strip 100 may be replaced by an elongated slot extending in a length direction of the sensor strip 100 to overlap a plurality of reaction chambers 130 in the same sensor strip 100. In some other configurations, except for a frame comprising portions of the fixing plate having formed thereon the insertion protrusions 400, some or substantially all portions of the fixing plate overlapping the sensor strips 100 may be removed. In this configuration, the removed portion of the fixing plate may overlap a plurality of reaction chambers 130 in different sensor strips 100. As configured, light may be transmitted through the bottoms of the reaction chambers 130 and the detection structures 10. Thus, the bottoms of the reaction chambers 130 and the detection structures 10 are made of light-transmitting materials such as polycarbonate, polyethylene terephthalate, polymethyl methacrylate, triacetyl cellulose, cycloolefin, polyarylate, polyacrylate, polyethylene naphthalate, polybutylene terephthalate or polyamide. However, these polymer materials are merely illustrative and the present invention is not necessarily limited thereto.

Referring to FIG. 10, each of the sensor strips 100 may include a plurality of detection structures 10, 10a, 10b, and 10c. The plurality of detection structures 10, 10a, 10b, and 10c may be vertically spaced apart from each other along the depth direction of the reaction chamber 130. The detection structure 10a may be arranged to face at least one other detection structure, e.g., detection structures 10b or 10c. The arrangement of the plurality of detection structures 10 increases the density (or concentration) of the immobilized biomolecule per unit volume of the sample. In some embodiments, the microstructures or nanostructures 11 may be formed on the surfaces of the one or more of the detection structures 10 and the immobilized biomolecule may in turn be arranged on the outer surface of the microstructures or nanostructures 11.

Referring to FIG. 10, the sensor strips 100 of the strip-type biosensor assemblies according to embodiments further include sample injection holes 300. The sample injection holes 300 may be recessed from one surface of the body 150 so as to be in communication with the reaction chambers 130. The sample is injected into the reaction chambers 130 through the sample injection holes 300 and the detection structures 10 are immersed in the sample.

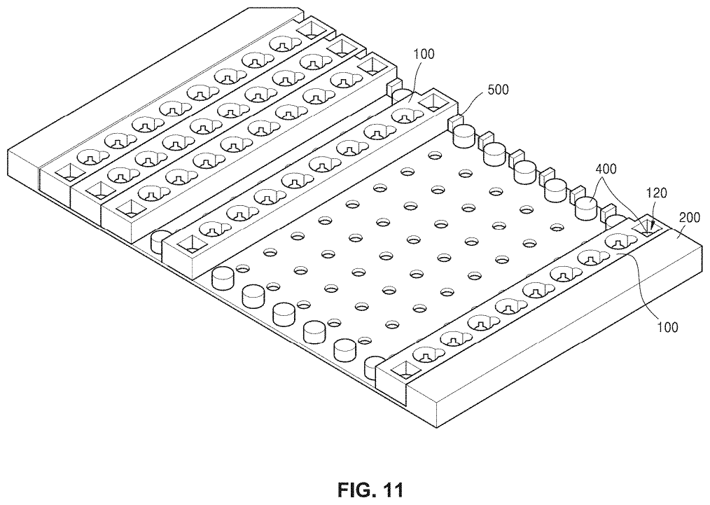

FIG. 11 is a perspective view of a strip-type biosensor assembly according to a further embodiment of the disclosed technology. The illustrated embodiments are similar in some aspects to the strip-type biosensor assembly described above with respect to FIGS. 8-10, and a detailed description of the similarities is not repeated herein for brevity. Referring to FIG. 11, the strip-type biosensor may further include fixing protrusions 500 to more firmly fix sensor strips 100 to a fixing plate 200. The fixing protrusions 500 protrude from one surface of the fixing plate 200 and are arranged at predetermined intervals from insertion protrusions 400. The distances between the fixing protrusions 500 and the insertion protrusions 400 are determined such that each of the fixing protrusions 500 is brought into contact with the outer surface of one end of the body 150 of the sensor strip 100 when the insertion protrusion 400 is inserted into an insertion hole 120. A corner of one end of the body 150 of the sensor strip 100 may be recessed inwardly. When the insertion protrusion 400 is inserted into the insertion hole 120, the recessed corner comes into close contact with the fixing protrusion 500, and as a result, the sensor strip 100 is firmly fixed to the fixing plate 200.

In the strip-type biosensor assemblies described above with respect to FIG. 10, the detection structures 10 are substantially similarly shaped and positioned substantially similarly with respect to a lateral position within the reaction chamber 130. Thus, when viewed in a top-down direction, the detection structures 10 substantially overlap each other. Various other embodiments are possible, as described in the following.

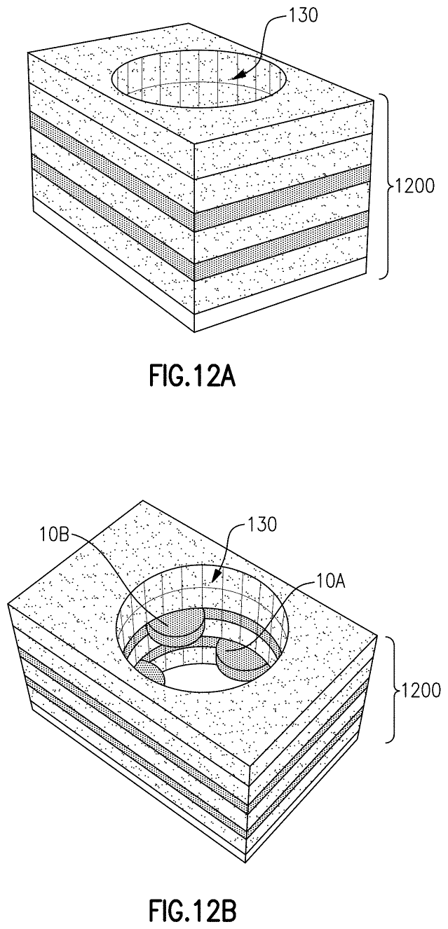

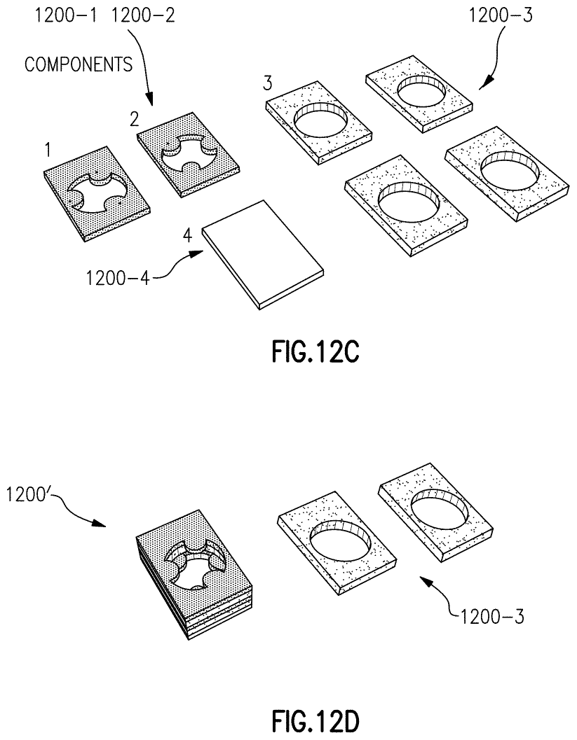

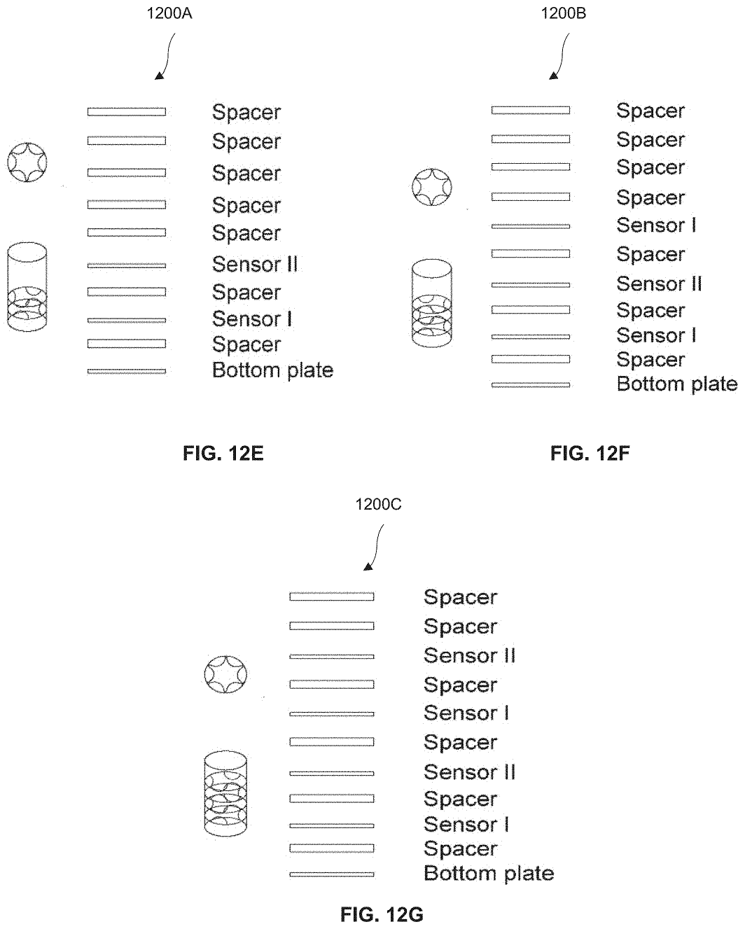

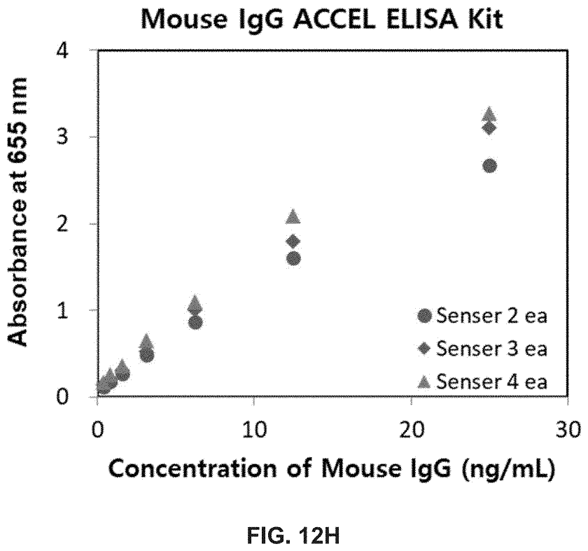

FIGS. 12A-12D, 13A-13D and 14A-14D illustrate embodiments of a strip-type biosensor according to some other embodiments, in which each of the one or more transparent detection structures comprises a protrusion extending from an inner surface of the respective one of the cavities.

FIGS. 12A and 12B illustrate perspective views at different angles of a portion of a sensor strip stack 1200 as part of a strip-type biosensor assembly, according to embodiments. For illustrative purposes, the portion comprises a reaction chamber or cavity 130 in which first and second detection structures 10A, 10B are formed. The sensor strip stack 1200 is formed by stacking a plurality of component layers. The sensor strip stack 1200 includes a bottom plate 1200-4, one or more detection structure layers 1200-1, 1200-2 and one or more spacer layers 1200-3. The one or more detection structure layers 1200-1, 1200-2 and the one or more spacer layers 1200-3 can be stacked in any suitable order. In the illustrated embodiment, the sensor strip stack 1200 includes, in a bottom-up direction, the bottom plate 1200-4, a first spacer layer 1200-3, a first detection structure layer 1200-1 including first detection structures 10A, a second spacer layer 1200-3, a second detection structure layer 1200-2 including second detection structures 10B, and third and fourth spacer layers 1200-3. Except for the bottom plate 1200-4, each of the detection structure layers 1200-1 and 1200-2, and each of the spacer layers 1200-3 comprise an opening formed therethrough. Unlike the spacer layers 1200-3, each of the detection structure layers 1200-1 and 1200-2 includes three detection structures formed at about 120° intervals along the circumference of the respective opening formed therethrough. FIG. 12C shows, for illustrative purposes, disassembled component layers that can be stacked to form the sensor strip stack 1200 illustrated in FIGS. 12A and 12B. As assembled, the portion of the sensor strip illustrated in FIGS. 12A, 12B has a reaction chamber or cavity 130 in which the first and second transparent detection structures 10A, 10B are formed. Each of the first and second transparent detection structures 10A, 10B comprises a plate structure extending laterally towards a central region of the respective one of the cavities. The first detection structures 10A of the detection structure layer 1200-1 and the second detection structures 10B of the detection structure layer 1200-2 are angularly rotated with respect to each other by about 60°.

For the purposes of more clearly illustrating the arrangement of the first and second detection structures 10A, 10B, FIG. 12D illustrates a portion of a partial sensor strip stack 1200′ without the top two spacer layers 1200-3. As illustrated, the one or more transparent detection structures comprise one or more first detection structures 10A formed as lateral protrusions by the pattern of the detection structure layer 1200-1 at a first vertical level in a depth direction (z-direction) of the reaction chamber, well or cavity 130, and one or more second detection structures 10B formed as lateral protrusions by the pattern of the detection structure layer 1200-2 at a second vertical level in the depth direction of the reaction chamber or cavity 130. Unlike the strip-type sensor assemblies described above with respect to FIGS. 8-11, the sensor assembly illustrated in FIGS. 12A-12D is configured such that the first detection structures 10A and the second detection structures 10B that are vertically separated by one or more spacer layers 1200-3 are laterally rotated relative to each other. As a result, at least a portion of each of the first detection structures 10A does not overlap any of the vertically adjacent second detection structures 10B in a lateral direction (x, y directions) perpendicular to a depth direction (z-direction) of the reaction chambers or cavities 130, such that when viewed in the z-direction, the non-overlapping portions of the first and second detection structures 10A, 10B that are rotationally offset with respect to each other are visible to a user. However, embodiments are no so limited and in other embodiments, the first detection structures 10A and the second detection structures 10B substantially overlap in the lateral direction. In yet other embodiments, the first detection structures 10A and the second detection structures 10B substantially overlap each other in the lateral direction.

Still referring to FIG. 12D, in the illustrated embodiment, the first and second detection structures 10A, 10B are regularly, e.g., periodically arranged, around the inner surface of the reaction chamber or cavity 130. For example, in the illustrated embodiment, adjacent ones of the first and second detection structures 10A, 10B are arranged to be separated by about 120° around the circumference of the reaction chamber or cavity 130. However, embodiments are not so limited and there may be any suitable number of first and second detection structures 10A, 10B such that the first and second protrusions 10A, 10B are arranged to be angularly separated by about 30°, 45°, 60°, 90°, 120° ′ or 180° around the circumference of the reaction chamber 130, to name a few example arrangements, or any angle θ defined by 360°/n, where n is an integer. In addition, in the illustrated embodiment, the first detection structures 10A and the second detection structures 10B are rotationally offset from each other by any fraction of 0. That is, when the angular separation between circumferentially adjacent ones of the first detection structures 10A and/or the second detection structures 10B is 0, the angular offset between the vertically adjacent ones of the first and second detection structures 10A and 10B may be expressed as θ/m, where m is any value greater than 1.

Advantageously, to provide additional volumes of liquid adjacent to active surfaces for enhanced diffusion and access of the active surfaces by the biomolecules, reagents and/or analytes in the sample to facilitate the ELISA processes, the one or more first detection structures 10A and the one or more second detection structures 10B are separated in a depth direction of the cavities by a spacer region formed by a spacer layer 1200-3 having a target thickness. Alternatively, one or more spacer layers 1200-3 may be formed between the vertically adjacent detection structure layers 1200-1, 1200-2. Similarly, one or more spacer layers 1200-3 having suitable thicknesses may be disposed above the detection structure layer 1200-2 and/or below the detection structure layer 1200-1. The suitable number of spacer layers and/or the thicknesses thereof can be customized to enhance the diffusional access to the active surfaces by the biomolecules and/or the reagents in the sample in contact therewith.

In addition, in a similar manner described above with respect to various embodiments, in addition to one or more of the main surfaces of the detection structures 10A, 10B, one or more of inner surfaces of respective ones of the reactive chambers or cavities 130 can serve as the active surfaces.

The stacked layer configuration of the strip-type sensor assemblies illustrated in FIGS. 12A-12D can offer many advantages. For example, by customizing the number of detection layers and structures, the overall reaction surface area can be customized. In addition, by customizing the thicknesses or the number of spacer layers between vertically adjacent detection structures, the volume of the sample immediately available for diffusional access of the active surfaces by the biomolecules or reagents can be customized.

In addition, because the first and second detection structures 10A, 10B are laterally offset relative to each other, the vertical distance between them can be reduced without negatively impacting diffusional access of vertically adjacent detection structures. As a result, compared to arrangements in which vertically adjacent detection structures laterally significantly overlap, a greater number of detection structures can be formed per unit depth of the reaction chamber or the cavity 130. In addition, a central region of each of the reaction chambers or cavities 130 that is unoccupied by the one or more transparent detection structures 10A, 10B is configured to easily receive the sample therein, e.g., using a tip of a pipette.

The customizability of the layers is further illustrated with respect to FIGS. 12E-12G. FIG. 12E illustrates a sensor strip stack 1200C which includes two detection structure layers, sensor I and sensor II. The sensor I may be arranged in a similar manner as the detection structure layer 1200-1, and the sensor II may be arranged in a similar manner as the detection structure layer 1200-2 as described above with respect to FIGS. 12A-12D. Each of the detection structure layers includes three detection structures formed at about 120° intervals along the circumference of an opening formed therethrough, in a similar manner as the arrangement illustrated in FIGS. 12A-12B. In addition, the detection structures of the vertically adjacent ones of the detection structure layers are angularly rotated with respect to each other by about 60°, and the adjacent ones of the detection structure layers are vertically separated by a spacer layer. FIGS. 12F and 12G illustrate sensor strip stacks 1200B and 1200C, respectively, which include three and four detection structure layers, respectively. Each of the detection structure layers includes three detection structures formed at about 120° intervals along the circumference of an opening formed therethrough, and vertically adjacent detection structure layers are separated by a spacer layer. The sensor strip stack 1200B includes two layers of sensor I (1200-1) interposed by the sensor II (1200-2). The sensor strip stack 1200C includes two layers of sensor I (1200-1) and two layers of the sensor II (1200-2) in an alternating arrangement.

Each of the sensor strip stacks 1200A-1200C illustrated in FIGS. 12E-12G can have the same total number of layers, and when the different layers have the same thickness, the sensor stacks can have substantially the same overall thickness (and the depth of the cavities) while having varying or customizable sensitivities. This customizability is illustrated in FIG. 12H, which shows a graph showing absorbance intensities at 655 nm for the same nominal concentration of mouse IgG. By increasing the number of detection structure layers and therefore the number of detection structures, the absorbance increases. For example, as illustrated, when the mouse IgG concentration is about 25 ng/mL, having three and four detection structure layers, where each detection layer comprises three detection structures, increases the absorbance by 16% and 23%, respectively, relative to having only two detection structure layers. Thus, as illustrated in FIGS. 12E-12G, the stacked layer configuration of the sensor strips can enable customization of the sensitivity of the strip-type sensor assemblies included in an ELISA kit.

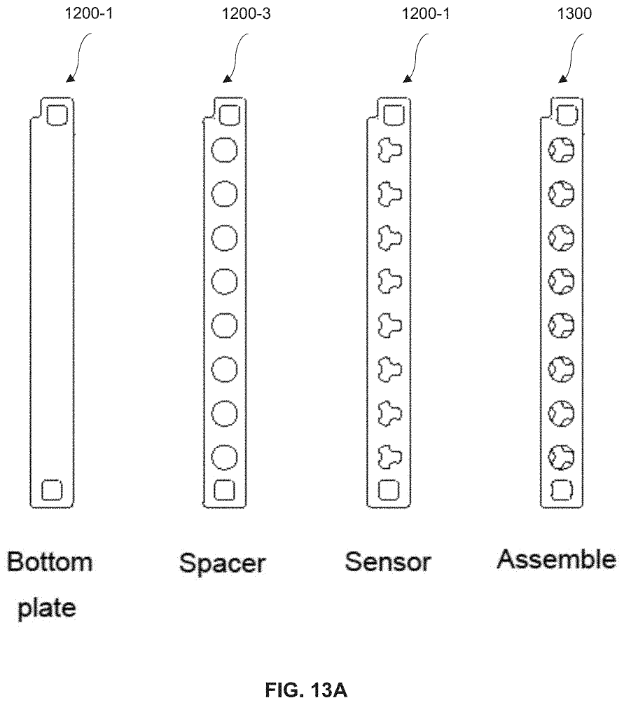

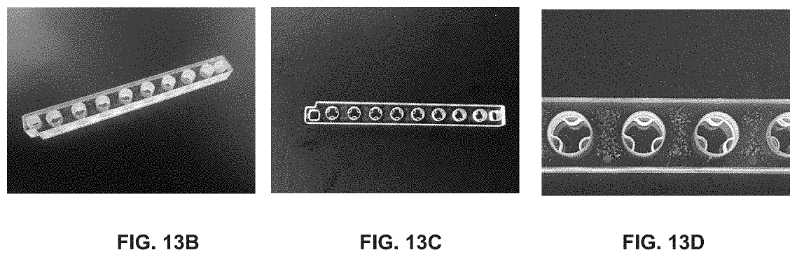

FIG. 13A illustrates top down views of the component layers of a sensor strip as part of a strip-type biosensor assembly similar to that described above with respect to FIGS. 12A-12G, including a bottom plate 1200-4, a detection structure layer 1200-1 including first or second detection structures 10A, 10B and one or more spacer layers 1200-1, which can be assembled into a sensor strip stack 1300. FIGS. 13B-13D illustrate photographic images of a perspective view, a top down view and a close up top down view, respectively, of the assembled sensor strip stack 1300 illustrated in FIG. 13A, when fully assembled.

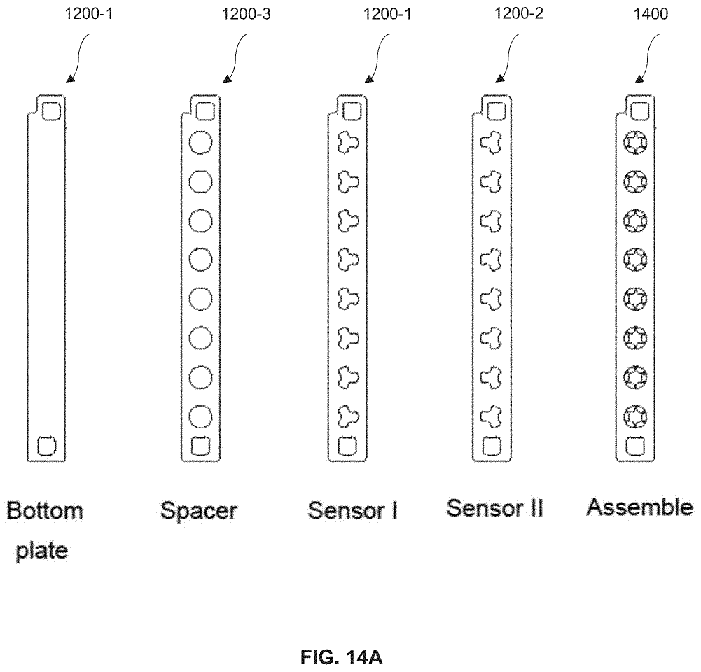

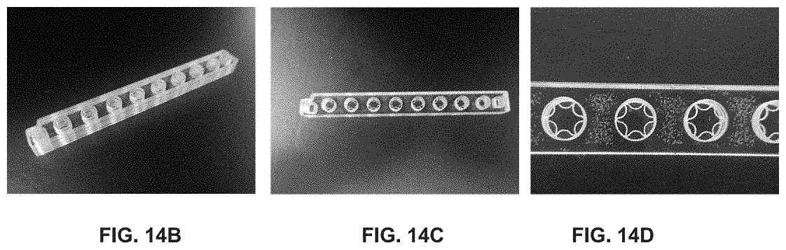

FIG. 14A illustrates top down views of the component layers of a sensor strip as part of a strip-type biosensor assembly similar to that described above with respect to FIGS. 12A-12G, including a bottom plate 1200-4, a detection structure layer 1200-1 including first detection structures 10A, a detection structure layer 1200-2 including second detection structures 10B and one or more spacer layers 1200-1, which can be assembled into a sensor strip stack 1400. FIGS. 14B-14D illustrate photographic images of a perspective view, a top down view and a close up top down view, respectively, of the assembled sensor strip stack 1400 illustrated in FIG. 14A, when fully assembled.

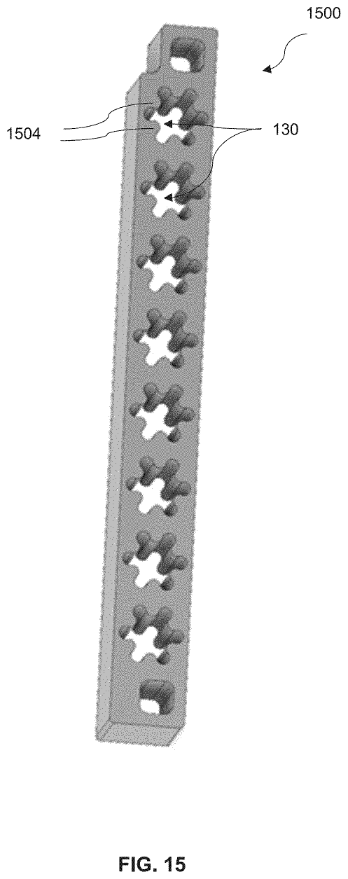

FIG. 15 is a perspective view of a sensor strip 1500 as part of a strip-type biosensor assembly according to a further embodiment of the disclosed technology. The sensor strip 1500 is similar in some aspects to the strip-type biosensor assemblies described above with respect to FIGS. 8-14D, and a detailed description of the similarities is not repeated herein for brevity. The sensor strip 1500 includes a plurality of reaction chambers, cavities or wells 130 in s similar manner as those described above with respect to FIGS. 8-14D. However, unlike the sensor strips described above with respect to FIGS. 8-14D, the reaction chambers 130 have corrugated sidewalls having a plurality of corrugations or detection structures 1504. In the illustrated embodiment, the corrugations 1504 are regularly, e.g., periodically arranged, around the inner surface of each of the reaction chambers or cavities 130. For example, in the illustrated embodiment, adjacent ones of the corrugations 1504 are arranged to be separated by about 60° around the circumference of the reaction chamber or cavity 130. However, embodiments are not so limited and there may be any suitable number of corrugations 1504 that are arranged to be angularly separated by any angle θ defined by 360°/n, where n is an integer. While in the illustrated embodiment, the corrugations 1504 are rounded corrugations, embodiments are not so limited, and the corrugations 1504 can have any suitable shape that includes a portion of a sphere, an ovoid, a pyramid (e.g. rectangular, triangular), a prism (e.g., rectangular, triangular), a cone, a cube, a cylinder, a plate, a disc, a wire, a rod, a sheet and fractals, to name a few. In addition, while in the illustrated embodiment, the corrugations 1504 are regularly spaced around the circumference of the wall of the reaction chamber 130, embodiments are not so limited and in some other embodiments, the corrugations 1504 may be irregularly spaced around the circumference of the wall of the reaction chamber 130. In the illustrated embodiment, the sensor strip 1500 is formed as a single piece article. However, embodiments are not so limited, and in some other embodiments, the sensor strip 1500 can be formed of a plurality of component layers, as described below.

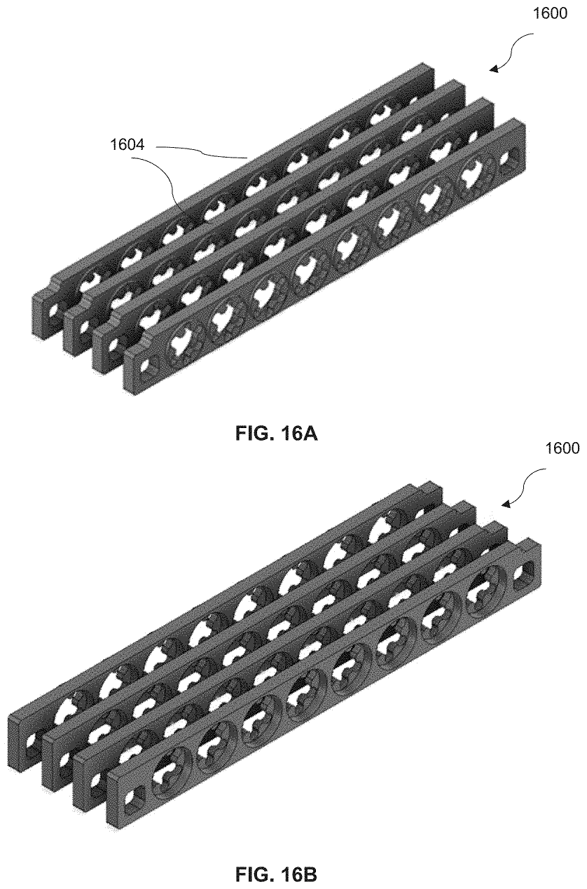

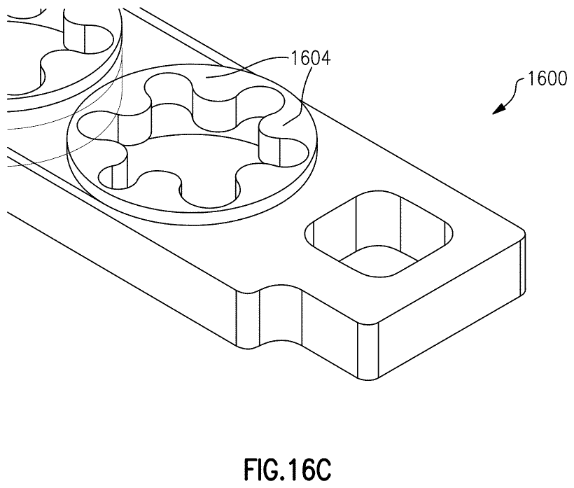

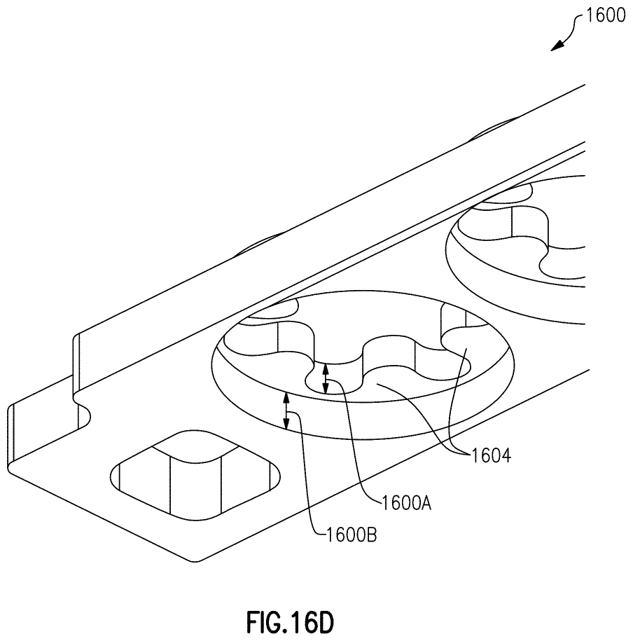

FIGS. 16A-16D are perspective views of a sensor strip stack 1600 as part of a strip-type biosensor assembly according to a further embodiment of the disclosed technology. The sensor strip stack 1600 is similar in some aspects to the strip-type biosensor assemblies described above with respect to FIGS. 8-15, and a detailed description of the similarities is not repeated herein for brevity. In a similar manner as described above with respect to 12A-14D, the sensor strip stack 1600 includes plurality of detection structure layers 1604. FIGS. 16A and 16B illustrate perspective views showing the upper and lower surfaces, respectively, of the detection structure layers 1604. FIGS. 16C and 16D illustrate perspective detailed views showing portions of the upper and lower surfaces, respectively, of the detection structure layers 1604. When assembled, the sensor strip stack 1600 additionally includes a bottom plate similar to the bottom plate 1300-4, which is not shown in FIGS. 16A-16D for clarity. When fully assembled, the sensor strip stack 1600 includes a plurality of reaction chambers, cavities or wells 130 having in s similar manner as those described above with respect to FIGS. 8-15.

Referring to the detailed bottom view of FIG. 16D, each of the detection structure layers 1600 has a first thickness portion 1600A and a second thickness portion 1600B. The first thickness portion 1600A comprises a detection layer portion having a plurality of corrugations 1604 that are regularly arranged, e.g., periodically arranged, around the inner surface of an opening formed therethrough. The shape and locations of the corrugations 1604 may be similar to the corrugations described above with respect to FIG. 15, and a detailed description is not repeated herein for brevity. Unlike the first thickness portion 1600A, second thickness portion 1600B comprises a spacer layer portion that does not have corrugations and is arranged in a similar manner as the spacer layer 1300-3 described above with respect to FIGS. 12A-12G. Thus, as integrated, the first and second thickness portions 1600A, 1600B are structurally analogous to a combination of a detection structure layer 1300-1/1300-2 and a spacer layer 1300-3 described above with respect to FIGS. 12A-12G. As arranged, the first thickness portion 1600A having the corrugations 1604 can be arranged similarly to, provide similar advantages as, and serve similar functions as the detection structure layer 1300-1/1300-2, and the second thickness portion 1600B can be arranged similarly to, provide similar advantages as, and serve similar functions as the spacer layer 1300-3 described above with respect to FIGS. 12A-12G.

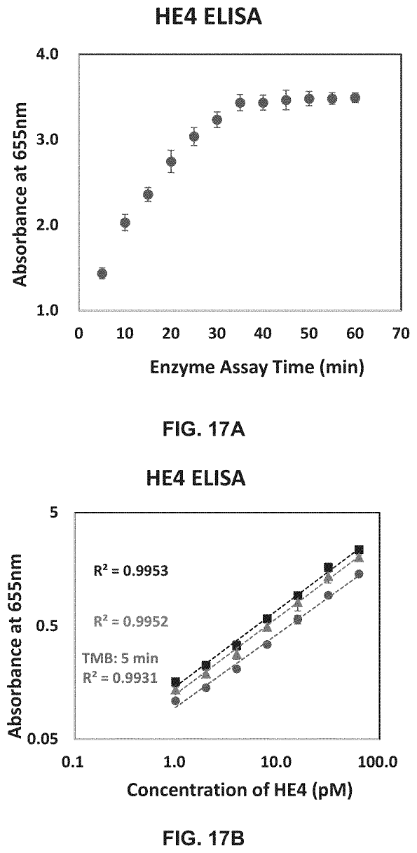

FIG. 17A is an experimental measurement of absorbance versus assay time for HE4 having a concentration of 62.5 pM using a TMB substrate solution, which was measured using a biosensor assembly, according to embodiments. The measurement shows the saturation point reached at about 30 minutes of assay time. FIG. 17B is an experimental measurement of absorbance versus concentration for HE4 having a concentration of 62.5 pM using a TMB substrate solution, which was measured using a biosensor assembly, according to embodiments. The results are summarized in the following TABLE 1.

TABLE 1

| | Immunoassay Time, Enzyme Assay Time | | | | | |
|---|---|---|---|---|---|---|
| HE4 | Sample 30 min, | | Sample 30 min, | | Sample 30 min, | |
| Concentration | TMB 5 min | | TMB 15 min | | TMB 30 min | |
| (pM) | AVE | % CV | AVE | % CV | AVE | % CV |
| Linearity (R² | 0.9931 | | 0.9952 | | 0.9953 | |
| value) | | | | | | |
| 0.0 | 0.0634 | 2.98 | 0.0759 | 2.18 | 0.0909 | 1.73 |
| 0.98 | 0.1092 | 4.18 | 0.1609 | 1.35 | 0.2058 | 1.79 |
| 1.95 | 0.1429 | 1.56 | 0.2248 | 5.42 | 0.3066 | 1.91 |
| 3.9 | 0.2090 | 3.77 | 0.3368 | 9.56 | 0.4756 | 0.29 |
| 7.8 | 0.3430 | 1.32 | 0.5799 | 6.51 | 0.8277 | 0.70 |
| 15.625 | 0.5729 | 8.61 | 0.9277 | 2.62 | 1.4138 | 4.41 |
| 31.25 | 0.9316 | 5.42 | 1.6440 | 8.79 | 2.2325 | 3.14 |
| 62.5 | 1.4381 | 4.41 | 2.3617 | 3.45 | 3.2347 | 2.84 |

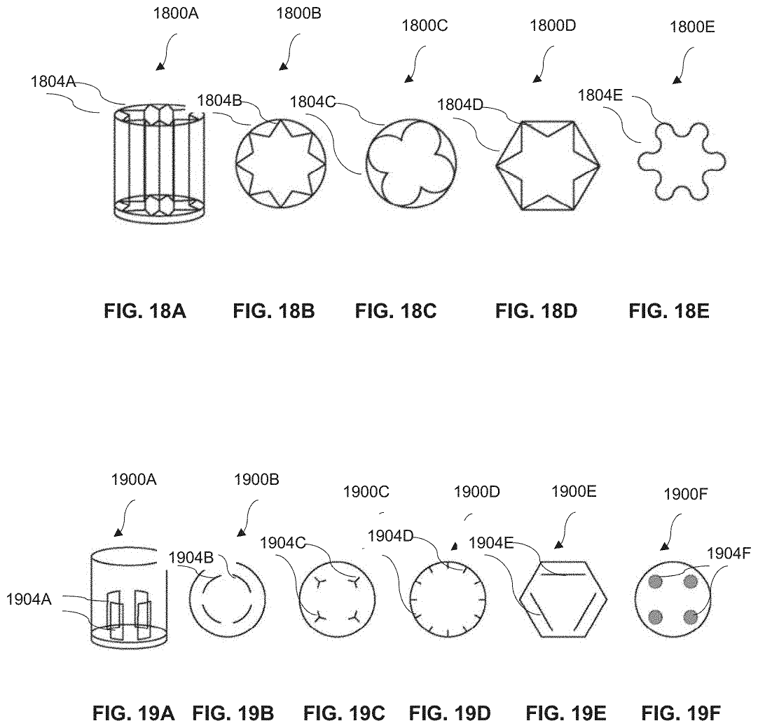

FIGS. 18A-18E illustrate portions of sensor strips 1800A-1800E, respectively, as parts of a strip-type biosensor assembly according to further embodiments of the disclosed technology. In particular, the portions of sensor strips 1800A-1000E illustrate alternative arrangement of detection structures extending from sidewalls of the reaction chambers. FIG. 18A illustrates a perspective view while FIGS. 18B-18E are plan-views. The portions of the sensor strips 1800A-1800E are similarly arranged in some aspects to the sensor strip described above with respect to FIG. 15, and a detailed description of the similarities is not repeated herein for brevity. As illustrated, the portions of the sensor strips 1800A-1800E include corrugations or detection structures 1804A-1804E, respectively, having various shapes and arrangements. In particular, the corrugations 1804A-1804E have various shapes including at least a partial prism (1804A, 1804B, 1804D), a partial cylinder, a wire or a rod (1804D) and sharpened edges (1804C), to provide a few examples.

According to various embodiments described herein having detection structures or corrugations extending inward toward a central region of the reaction chamber or cavity, the detection structures or corrugations have a peak distance from the sidewall of the reaction chamber that is configured to provide varying degrees of enhancement in the surface area available for reaction. The peak distance can be, e.g., 0.5 mm to 1 mm, 1 mm to 1.5 mm, 1.5 mm to 2.0 mm, 2.0 mm to 2.5 mm, 2.5 mm to 3.0 mm, 3.0 mm to 3.5 mm, or a value in a range defined by any of these values.

FIGS. 19A-19F illustrate portions of sensor strips 1900A-1900F, respectively, as parts of a strip-type biosensor assembly according to further embodiments of the disclosed technology. In particular, the portions of sensor strips 1900A-1900F illustrate alternative arrangement of detection structures extending from a bottom plate. FIG. 19A illustrates a perspective view while FIGS. 19B-19F are plan-views. The portions of the sensor strips 1900A-1900F are similarly arranged in some aspects to the sensor strip described above with respect to FIGS. 15 and 18A-18E, and a detailed description of the similarities is not repeated herein for brevity. Unlike the embodiments illustrated with respect to FIGS. 15 and 18A-18E, in which the corrugations or detection structures extend radially inward from the sidewalls of the reaction chamber or cavity 130, in the illustrated embodiments, the detection structures 1904A-1904F are attached to and extend from the bottom plate. As illustrated, the portions of the sensor strips 1900A-1900F include detection structures 1904A-1904F, respectively, having various shapes and arrangements. In particular, the detection structures 1904A-1904F have various shapes including a vertical planar strip, (1904A, 1904D, 1904F), a vertical curved strip (1904B), a cylinder, wire or rod (1804F) and joined strips (1904C), to provide a few examples. The detection structures 1904A-1904F can be detached from or attached to the sidewalls of the reaction chambers or cavities 130.

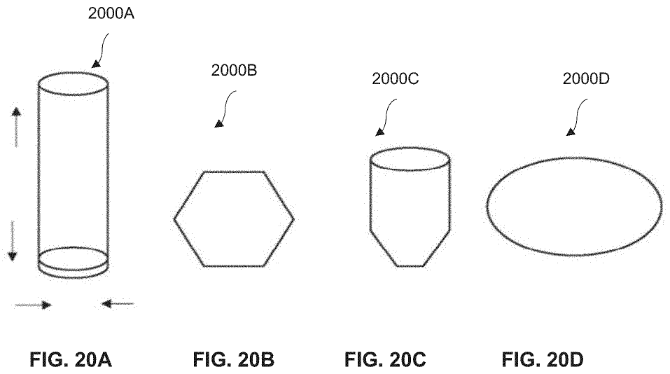

FIGS. 20A-20D illustrate portions of sensor strips 2000A-2000D, respectively, as parts of a strip-type biosensor assembly according to further embodiments of the disclosed technology. FIG. 20A illustrates a perspective view while FIGS. 20B-20D are plan-views. In particular, the portions of the sensor strips 2000A-2000D illustrate alternative shapes of the reaction chambers, cavities or wells 130. The portions of the sensor strips 2000A-2000D are similarly arranged in some aspects to the sensor strip described above with respect to FIGS. 8-19F, and a detailed description of the similarities is not repeated herein for brevity. As illustrated, at least portions of the reaction chambers 130 can have various shapes, including a circular or ovular cylinder (2000A, 2000D), a polygonal prism (2000B) or a cone (2000C), to provide a few examples. The reaction chambers 130 can have various dimensions, including height, width and volume, according to different implementations. The reaction chambers 130 can be radially symmetric or asymmetric, and can have the same or different top and bottom dimensions, according various implementations.

### Surface to Volume Ratios of Reaction Chambers

In each of the embodiments of the strip-type sensor assemblies described herein, e.g., with respect to FIGS. 8 to 20D, various surface modifications described above with respect to cuvette-type sensor assemblies described above, e.g., with respect to FIGS. 3A-3E. In addition, various parameters including, e.g., the number, thicknesses, surface areas and active surface areas of the detection structures, volume capacities of the reaction chambers, cavities or wells, distance between adjacent surfaces and a ratio of combined active surface areas to the volume of liquid held by the cavities, can have one or more values described herein. The inventors have realized that, by controlling these and other various parameters, the reaction surface area participating in an ELISA reaction can be controlled and optimized. TABLE 2 below is an example calculation for a reaction chamber having an arrangement similar to those described above with respect to FIGS. 12F-12G.

TABLE 2

| | | | | | Ratio of |
|---|---|---|---|---|---|
| | | | | | Contacted |
| No of | | Minimum | | | Surface to |
| Detection | | Surface Area | | Minimum | Sample |
| Structure | Total Surface | Contacted by | Total Volume | Sample | Volume |
| Layers (N) | Area (mm²) | Sample (mm²) | (mm³) | Volume (mm³) | (mm²/μL) |
| 1 | 105.84 | 67.03 | 83.34 | 34.65 | 1.93 |
| 2 | 150.73 | 111.93 | 117.99 | 69.30 | 1.61 |
| 3 | 195.63 | 156.82 | 152.64 | 103.95 | 1.51 |
| 4 | 240.53 | 201.72 | 187.29 | 138.60 | 1.45 |
| N | 60.94 + (44.90N) | 22.13 + (44.90N) | 48.69 + (34.65N) | 34.65N | 1.29 + 0.64/N |

The calculations in TABLE 2 are for a cylindrical reaction chamber having a diameter of 5 mm and detection structures that are disc-type structures having a radius of 1.1 mm. The minimum surface area contacted by the sample and the minimum volume of the sample correspond to the minimum amount of sample needed to completely immerse the detection structures. As a comparison, for a commercially available cylindrical reaction chamber having a height of 10.75 mm and diameter of 6.66 mm, and for a sample volume of 100 μL, the contacted surface area is about 95.93 mm², corresponding to the ratio of the contacted surface area to the sample volume of less than 1. In comparison, the reaction chambers having detection structures therein according to embodiments provide much higher values of the ratio of the contacted surface area to the sample volume, as shown in TABLE 2.

TABLE 3 below are example calculations of the reaction area that can be soaked in a sample as a function of the diameter of a cylindrical reaction chamber for 100 μL of sample volume.

TABLE 3

| Diameter (mm) | Soaked Area (mm²) |
|---|---|
| 3 | 140.40 |
| 4 | 112.57 |
| 5 | 99.63 |
| 6 | 94.94 |
| 7 | 95.63 |
| 8 | 100.27 |
| 9 | 108.06 |

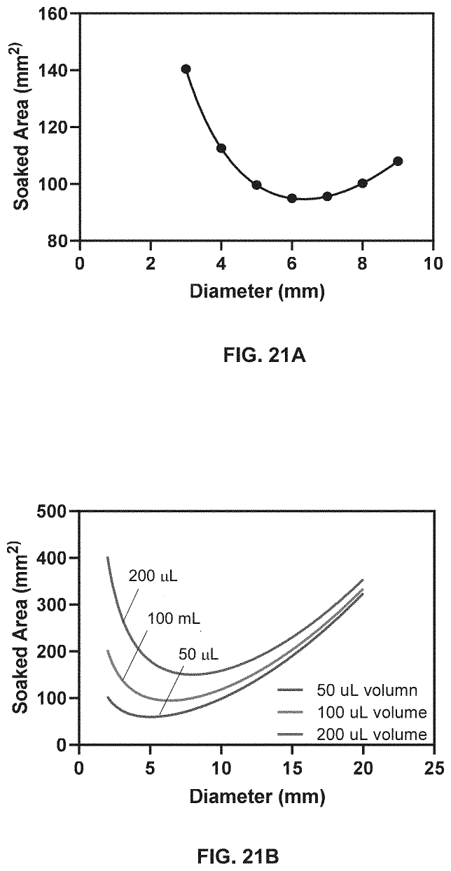

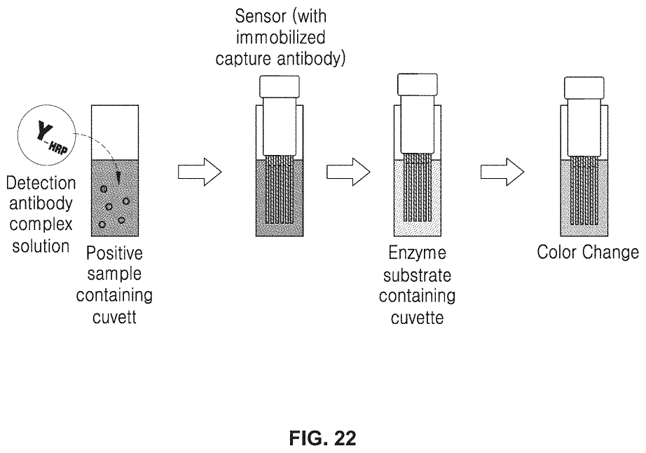

The calculated relationship between the soaked area and the reaction chamber diameter is graphically illustrated in FIG. 21A. Based on the results, the inventors have determined that, for a given volume of the sample, a diameter of the reaction chamber corresponding to a minimum soaked area exists. For the sample volume of 100 μL in the illustrate example, a diameter of about 6 mm results in approximately the lowest soaked area, whereas the soaked area increases in nonlinear inverse proportion to the diameter below 6 mm. Accordingly, an ELISA reaction rate accordingly increases when the diameter is below about 6 mm.

FIG. 21B illustrates the calculated relationships between the soaked area and the reaction chamber diameter for various sample volumes. As illustrated, for sample volumes between about 50 μL and 200 μL, the diameter of the reaction chamber below which the active surface area of the reaction chamber or the soaked area increases rapidly is between about 7 mm and about 5 mm. For various sample volumes and the respective diameters below which the soaked areas increase rapidly, the soaked area can have any values between about 50 mm² and about 400 mm². In accordance with these observations, the soaked surface area-to-volume ratio of a reaction chamber according to embodiments is between about 0.25 mm²/μL to about 8 mm²/μL. For example, the soaked surface area-to-volume ratio of a sample in the reaction chamber or the reaction chamber itself can be between about 0.25 mm²/μL to 1 mm²/μL, 1 mm²/μL to 2 mm²/μL, 2 mm²/μL to 3 mm²/μL 3 mm²/μL to 4 mm²/μL, 4 mm²/μL to 5 mm²/μL, 5 mm²/μL to 6 mm²/μL, 6 mm²/μL to 7 mm²/μL, 7 mm²/μL to 8 mm²/μL, or a value in a range defined by any of these values.

### Immunoassay Methods Using Sensitivity- and Diffusion-Enhanced Sensor Assemblies

The sensor assemblies according to various embodiments described herein can advantageously be used to perform one or more of various immunoassays, e.g., ELISA processes, according to various embodiments. According to various embodiments, a method of conducting an ELISA comprises providing an ELISA kit according to any one of embodiments described herein. The ELISA kit includes one or more reagents, biomolecules and/or analytes for an ELISA and a sensor assembly adapted for the ELISA according to various embodiments described above. The method additionally includes conducting an ELISA reaction within the optically transparent container, e.g., a cuvette or one or more cavities of a strip sensor, as described above. The method additionally includes: providing a solution comprising a target analyte and a marker-labeled detection reagent that is configured to specifically bind to the target analyte; immobilizing on the active surfaces of the sensor assembly a capturing reagent configured to specifically bind to a target analyte; at least partially immersing the active surfaces in the solution to cause the target analyte to be specifically bound to the capturing reagent and to the marker-labeled detection reagent; and detecting the target analyte specifically bound to the capturing reagent and to the marker-labeled detection reagent.

According to various embodiments, a method of conducting an ELISA comprises providing an ELISA well, wherein the ELISA well comprises: a transparent container, and one or more detection structures within the optically transparent container, wherein the one or more detection structures have active surfaces configured to allow an antibody to be bound thereto, wherein the one or more detection structures provide a ratio of a combined surface area of the one or more detection structures to a volume of the liquid between about 0.25 mm² per microliter and about 8.0 mm² per microliter, or any value disclosed herein. The method additionally comprises conducting an ELISA with the optically transparent container, wherein only a single wash is involved in the ELISA.

Different types of ELISA used in the art include direct ELISA, indirect ELISA, sandwich ELISA and competitive ELISA, to name a few.

In a direct ELISA, the antigen is immobilized on the surface of a multi-well plate and detected with an antibody configured to specifically bind to the antigen and directly conjugated to detection molecules such as horseradish peroxidase (HRP).

In an indirect ELISA, similar to direct ELISA, the antigen is immobilized to the surface of the multi-well plate. However, a two-step process is used for detection whereby a primary antibody specific for the antigen binds to the target, and a labeled secondary antibody against the host species of the primary antibody binds to the primary antibody for detection. The method can also be used to detect specific antibodies in a serum sample by substituting the serum for the primary antibody.

In a sandwich ELISA (or sandwich immunoassay), two antibodies, sometimes referred to as matched antibody pairs, specific to the antigen are used. One of the antibodies is coated on the surface of the multi-well plate and used as a capture antibody to facilitate the immobilization of the antigen. The other antibody is conjugated and facilitates the detection of the antigen.

In a competitive ELISA, also referred to as inhibition ELISA or competitive immunoassay, the concentration of an antigen is measured by signal interference. The sample antigen competes with a reference antigen for binding to a specific amount of labeled antibody. The reference antigen is pre-coated on a multi-well plate. The sample is pre-incubated with labeled antibody and added to the wells. Depending on the amount of antigen in the sample, more or less free antibodies will be available to bind the reference antigen. This means the more antigen there is in the sample, the less reference antigen will be detected and the weaker the signal. The labeled antigen and the sample antigen (unlabeled) compete for binding to the primary antibody. The lower the amount of antigen in the sample, the stronger the signal due to more labeled antigen in the well.

According to various embodiments, the ELISA protocol and/or ingredients described herein can be for an indirect ELISA, a direct ELISA with streptavidin detection, a sandwich ELISA, a competition ELISA, and/or a sandwich ELISA with strep-biotin detection.

In some embodiments, the kit can include one or more of a: coating buffer, a blocking buffer (such as PBS, optionally with 1% BSA)), and a wash buffer (such as PBS with 0.05% v/v Tween-20. In some embodiments, the kit can further include a substrate solution (such as TMB Core+(BHU062) or pNPP (BUF044)) and a stop solution (such as 0.2M H₂SO₄ or 1M NaOH).

In some embodiments, the method of performing the ELISA can include one or more of the following: coating the surfaces of the detection structures and/or the surfaces of the wells with antigen solution, optionally washing the plates in water, adding blocking solution and washing the plates, adding unconjugated detection antibody and wash plates, adding enzyme-conjugated secondary antibody and wash plates, adding substrate solution and allowing the reaction to occur and then reading absorbance from the cuvette or well. This can be for an indirect ELISA.

In some embodiments, the method of performing the ELISA can include one or more of the following: coating the wells with antigen solution, optionally washing the plates in water, adding blocking solution and washing the plates, adding sample to the wells, adding biotin-conjugated detection antibody to each well (optionally washing), adding enzyme-conjugated streptavidin to the wells (optionally washing), adding substrate solution to the wells (or cuvette), and then reading absorbance. This can be for a direct ELISA.

In some embodiments, direct ELISA is comprised of the following steps: (i) coating a solid phase with an antigen dissolved in a coating buffer; (ii) incubating the solid phase from Step (i) with a blocking reagent for 1 hour to block non-specific binding sites on the solid phase; (iii) optionally washing the solid phase from Step (ii) three times with PBS or PBST for 1 min each; (iv) incubating the solid phase from Step (iii) with a primary detection agent which binds to the antigen; (v) optionally washing the solid support from Step (iii) five times for 1 min each in PBS or PBST to remove the non-specifically bound primary detection agent; and (vi) using a detection system such as UV, fluorescence, chemiluminescence or other detection methods to detect the bound primary detection agent. The primary detection agent can be, without limitation, a detection agent linked (coupled) to a fluorescent dye, or a reporter enzyme such as alkaline phosphatase (AP) or horseradish peroxidase (HRP), which can convert a colorless substrate to a colored product whose optical densities can be measured on an ELISA plate reader at target wavelengths.

In some embodiments indirect ELISA comprises of the following steps: (i) coating a solid phase with an antigen dissolved in a coating buffer; (ii) incubating the solid phase from Step (i) with a blocking reagent for 1 hour to block non-specific binding sites on the solid phase; (iii) optionally washing the solid phase from Step (ii) three times with PBS or PBST for 1 min each; (iv) incubating the solid phase from Step (iii) with a primary detection agent diluted in a solution for 1 hour; (v) optionally washing the solid support from Step (iv) three times for 1 min in PBS or PBST to remove the non-specifically bound primary detection agent; (vi) incubating the solid support from step (v) with a secondary detection agent diluted in a solution for 1 hour; (vii) optionally washing the solid support from Step (vi) five times for 1 min each in PBS or PBST to remove the non-specifically bound secondary detection agent; and (viii) using a detection system such as UV, fluorescence, chemiluminescence or other methods to detect the bound secondary detection agent. The secondary detection agent binds the primary detection agent. The secondary detection agent can be, without limitation, a detection agent linked (coupled) to a reporter enzyme such as alkaline phosphatase (AP) or horseradish peroxidase (HRP), which can convert a colorless substrate to a colored product whose optical densities can be measured on an ELISA plate reader at target wavelengths.

In some embodiments, the direct ELISA procedure involves at least three incubation steps: the first is incubation between the solid support and the antigen; the second is incubation between the solid support and the blocking reagent; and the third one is incubation between the solid support and the primary detection agent. The incubation step can be a two-phase reaction and involves the binding reaction between the antigen on the solid support and the detection agent.

In some embodiments, the indirect ELISA procedure involves at least four incubation steps: the first is incubation between the solid support and an antigen; the second is incubation between the solid support and the blocking reagent; the third one is incubation between the solid support and the primary detection agent; and the fourth is incubation between the solid support and the secondary detection agent. The incubation step can be a two-phase reaction and involves the binding reaction between the antigen on the solid support and the detection agent.

In some embodiments, for direct ELISA, the first incubation step, antigen coating, takes at least 2 hours and each other incubation step takes about 1 hour.

In some embodiments, for indirect ELISA, the first incubation step, antigen coating, takes at least 2 hours and each other incubation step takes about 1 hour.

In some embodiments, a cell-based ELISA (C-ELISA) is a moderate throughput format for detecting and quantifying cellular proteins including post-translational modifications associated with cell activation (e.g., phosphorylation and degradation). Cells are plated, treated according to experimental requirements, fixed directly in the wells, and then permeabilized. After permeabilizing, fixed cells are treated similar to a conventional immunoblot, including blocking, incubation with a first antibody, washing, incubation with a second antibody, addition of chemiluminescent substrates and development.

In some embodiments, ELISA is carried out by overnight coating the activated wells with antigen at 4 degrees C., blocking the wells in 2 hours at 37 degrees C. followed by antibody and conjugate binding at 37 degrees C. for 2 h each and color development that is enzyme-substrate reaction at room temperature for 5 minutes followed by reading absorbance.

In some embodiments, the ELISA kit can be one or more of an: Acetylcholine ELISA Kit, AGE ELISA Kit, CXCL13 ELISA Kit, FGF23 ELISA Kit, HMGB1 ELISA Kit, iNOS ELISA Kit, LPS ELISA Kit, Malondialdehyde ELISA Kit, Melatonin ELISA Kit, NAG ELISA Kit, OVA ELISA Kit, Oxytocin ELISA Kit, PGE2 ELISA Kit, PTHrP ELISA Kit, S100b ELISA Kit, Tenascin C ELISA Kit, VEGF-B ELISA Kit, and/or Versican ELISA Kit.

In some embodiments, an ELISA can be performed under a first set of conditions that allow selective and/or specific binding of the antibody or other binding molecule to the target or antigen. The ELISA can then continue under either the same set of conditions or have those conditions changed during the enzymatic step of the technique. This can allow the enzymatic process more variation in process parameters. In some embodiments, an antibody (as shown in 1A) is employed to immobilize the antigen. In some embodiments, other proteins or structures (a binding molecule, such as receptor molecules or enzymes, etc.) can be immobilized, as long as they are still capable of binding to the target molecule. Thus, as will be appreciated by one of skill in the art, while the term ELISA is used throughout, the methods and devices provided herein are not limited to “immuno” assays and can instead employ other binding molecules in place of the immuno (e.g., antibody) component for any of the embodiments provided herein. This applies to all appropriate embodiments provided herein. In some embodiments, the ELISA employed is one or more of a direct ELISA, an indirect ELISA, a sandwich ELISA, a competitive ELISA and/or a reverse ELISA.

In some embodiments, the method involves a first binding solution to allow for the ideal binding selectivity, and a second solution for the enzymatic component of the assay. In some embodiments, the buffers in the solutions are one in the same. In some embodiments, the buffers are changed throughout the process (and can vary in salt concentration or type of mono or divalent salts present, or other ingredients as well). In some embodiments, the temperature during the binding phase is designed for binding while the temperature during the enzymatic phase is designed for enzymatic activity. In some embodiments, the temperatures are the same or substantially the same. In some embodiments, the temperatures differ, but still allow for continued binding during the enzymatic phase. In some embodiments, the temperature and/or solution ingredients change between binding and enzymatic phases to the extent that some or much, or even all of the target may dissociate from the binding molecule. However, this can be addressed by keeping the target bound to the binding molecule during the binding phase and through any wash out phase (if present), and then retaining the solution for the enzymatic phase in the well or same volume of liquid. In other embodiments, the target remains bound to the antigen binding molecule throughout the process.

In the following, a description of an example homogeneous immunoassay method, referred to herein as a one-step immunoassay method, is provided for antigen detection using the biosensor according to some embodiments.

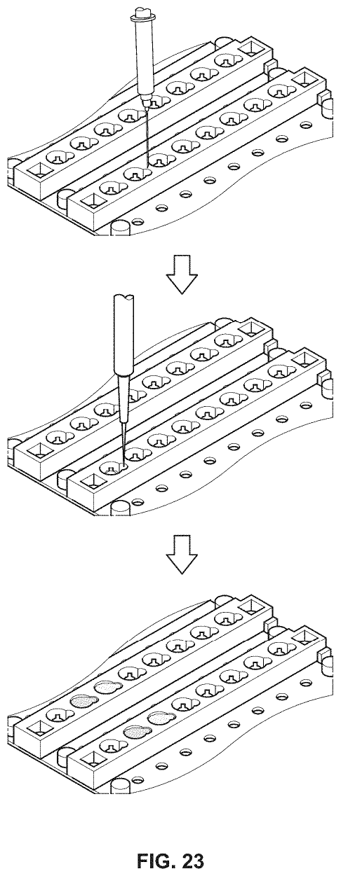

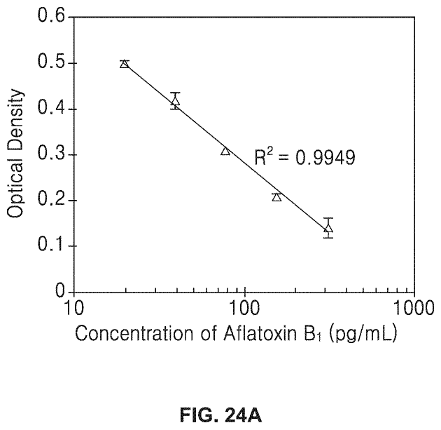

FIG. 22 is a flow diagram illustrating a method for analyzing a sample using the cuvette-type biosensor according to some embodiments and FIG. 23 is a flow diagram illustrating a method for analyzing a sample using the strip-type biosensor according to some embodiments.

First, as illustrated in FIG. 22, a one-step immunoassay method for antigen detection using the cuvette-type biosensor according to some embodiments includes (a) preparing a sample containing a target analyte (e.g., an antigen) and a detection biomolecule complex solution (e.g. a detection antibody complex solution) containing a marker-labeled detection biomolecule (e.g., a detection antibody), (b) sequentially immersing the detection structures surface bound with the immobilized biomolecule (e.g., a capture antibody) capable of binding to the target analyte in any order in the sample and the detection biomolecule complex solution or immersing the detection structure surface bound with the immobilized biomolecule in a mixture solution of the sample and the detection biomolecule complex solution, and (c) measuring the absorbance of the reaction products.

In step (b), the immobilized biomolecule, the target analyte, and the marker-labeled detection biomolecule react with one another. Approximately 15-30 minutes after initiation of the reactions, the detection structures are immersed in the cuvette in which an enzyme substrate is accommodated and the absorbance of the cuvette is measured. After completion of step (b), the detection structures may be washed to remove unreacted target analyte and/or marker-labeled detection biomolecule and the detection structures may be immersed in the cuvette in which the enzyme substrate is accommodated.

When the strip-type biosensor is used, in step (b), the sample and the detection biomolecule complex solution are sequentially injected in any order through the sample injection holes or mixture of the sample and the detection biomolecule complex solution may be injected through the sample injection holes. In step (c), an enzyme substrate is injected and the absorbance of the reaction products is measured.

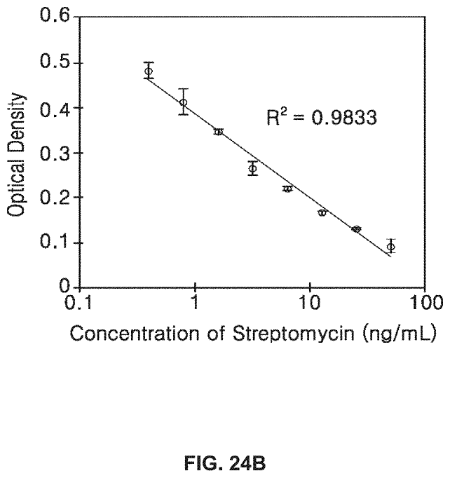

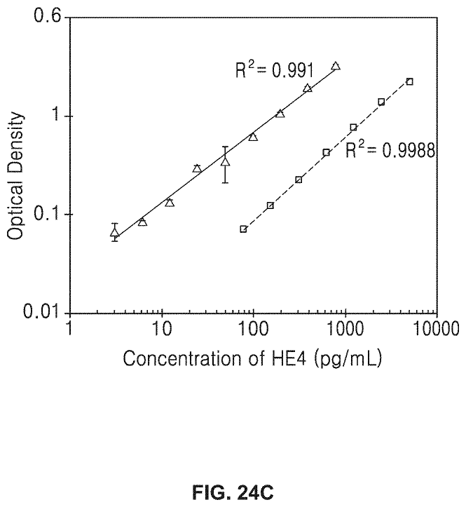

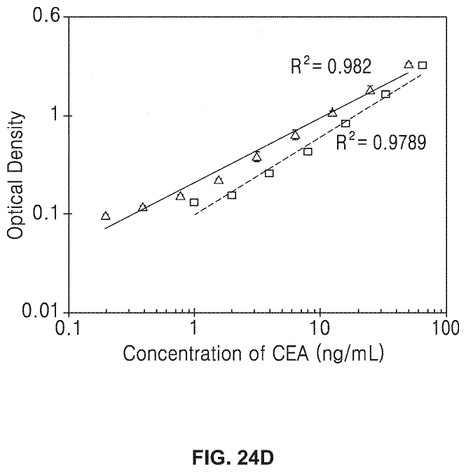

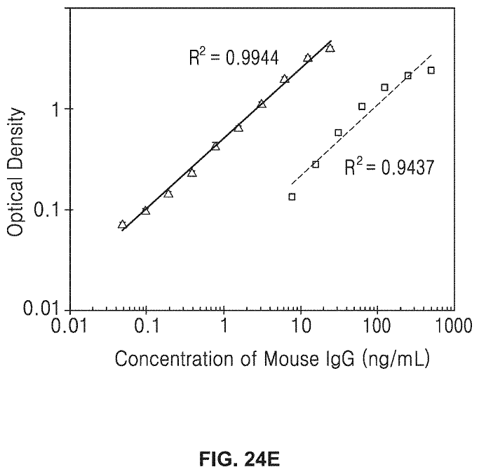

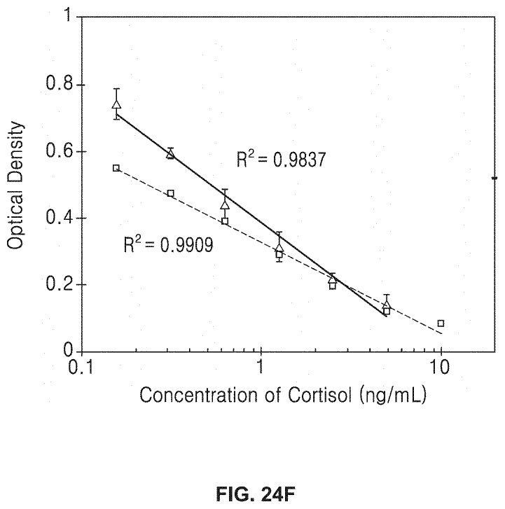

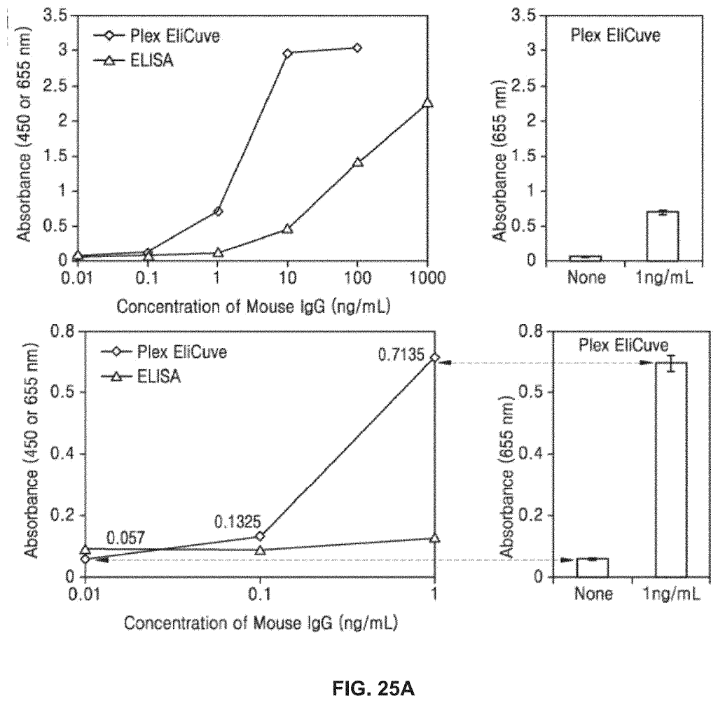

Aflatoxin B 1, streptomycin, human epididymis protein 4 (HE4), carcinoembryonic antigen (CEA), mouse IgG, and cortisol and (concentration-dependent) were used as target analytes and the absorbances of reaction products with the target analytes at different concentrations were measured. The results are shown in FIGS. 24A to 24F.

Aflatoxin B1 at concentrations ranging from 19.53 to 312.5 pg/mL was detected within 45 min (see FIG. 24A). Streptomycin at concentrations ranging from 0.39 to 50 ng/mL was detected within 30 min (see FIG. 24B).

To determine whether the sensitivity of the inventive biosensor was improved, a currently commercially available biosensor (ELISA kit, R&D Systems) was used to analyze HE4, CEA, mouse IgG, and cortisol. The absorbances measured from the inventive biosensor and the commercial biosensor are indicated by solid lines and dashed lines in FIGS. 24C to 24F, respectively.

The inventive biosensor succeeded in detecting HE4 at concentrations ranging from 6.1 to 390 pg/mL within 30 min whereas the commercial biosensor detected the same target analyte at concentrations ranging from 78 to 5,000 pg/mL within 4 h (see FIG. 24C).

The inventive biosensor succeeded in detecting CEA at concentrations ranging from 0.2 to 390 ng/mL within ˜15-30 min whereas the commercial biosensor detected the same target analyte at concentrations ranging from 1 to 65 ng/mL within 90 min (see FIG. 24D).

The inventive biosensor succeeded in detecting mouse IgG at concentrations ranging from 0.05 to 25 ng/mL within 30 min whereas the commercial biosensor detected the same target analyte at concentrations ranging from 7.8 to 500 ng/mL within 120 min (see FIG. 24E).

The inventive biosensor succeeded in detecting cortisol at concentrations ranging from 0.15 to 5 ng/mL within 45 min. whereas the commercial biosensor detected the same target analyte at concentrations ranging from 0.15 to 10 ng/mL within 180 min. (see FIG. 24F).

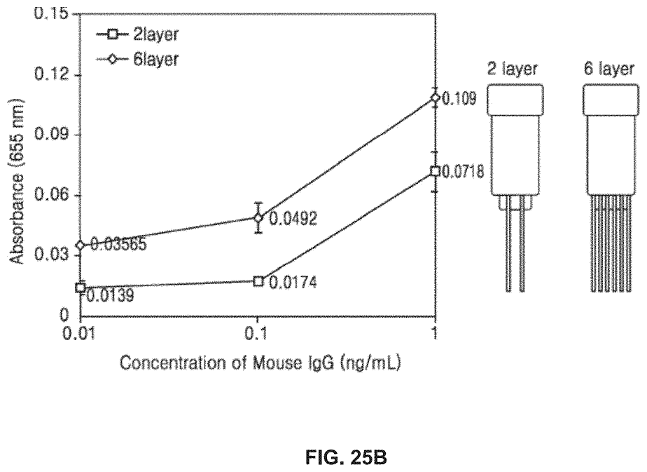

FIGS. 25A and 25B are experimental measurements of absorbances of reaction products with various analyte antibodies at different concentrations, which were measured using a biosensor assembly according to embodiments. As described in various embodiments above, the active areas or reaction areas of the various configurations of biosensor assemblies according to embodiments are much larger than that of the conventional ELISA plates, for comparable sample volumes. As compared with conventional ELISA plates, embodiments have much larger number of the immobilized substances (capturing molecules or receptors) that can react with targets. In this regard, the biosensor enables an assay with reduced Hook effect (enhanced assay sensitivity) as well as with increased reaction rate (reduced assay time). This is illustrated with respect to FIGS. 25A and 25B, in which by stacking a plurality of detection structures, the concentration of a receptor or antibody reactive with a protein, e.g., an antigen, is substantially enhanced. For example, as shown in FIG. 25B, the biosensor with six detection structures detects IgG with higher sensitivity than that with two detection structures. As described above, the higher sensitivity for the biosensor with a larger number of the detection structure can be attributed to the higher density of immobilized biomolecules and/or analytes (e.g., capturing molecules or receptors) available in the defined volume, as well as enhanced diffusion thereof.

Taken together, these results show that the biosensor according to some embodiments has greatly improved sensitivity and can detect a target analyte in a short time.

### Example Embodiments

1. An enzyme linked immunosorbent assay (ELISA) kit, comprising:

one or more reagents for an ELISA and a sensor assembly adapted for the ELISA, wherein the sensor assembly comprises:

a transparent container having one or more cavities formed therein,

a plurality of active surfaces disposed in each of the one or more cavities and configured for immobilizing a reagent thereon, and

one or more transparent detection structures disposed in each of the one or more cavities, wherein each of the transparent detection structures comprises one or more main surfaces that provide one or more of the active surfaces,

wherein the one or more cavities are configured to be filled with a liquid such that each of the transparent detection structures are at least partially submerged therein, wherein a ratio of a combined surface area of the transparent structures contacted by the liquid to a volume of the liquid exceeds about 0.25 mm² per microliter, and

wherein each of the active surfaces is separated from an immediately adjacent one of the active surfaces by a distance exceeding about 500 microns.

2. An enzyme linked immunosorbent assay (ELISA) kit, comprising:

one or more reagents for an ELISA and a sensor assembly adapted for the ELISA, wherein the sensor assembly comprises:

a transparent container having one or more cavities formed therein,

a plurality of active surfaces disposed in each of the one or more cavities and configured for immobilizing a reagent thereon, and

one or more transparent detection structures disposed in each of the one or more cavities, wherein each of the transparent detection structures comprises one or more main surface that provide one or more of the active surfaces,

wherein the one or more cavities are configured to be filled with a liquid such that each of the transparent detection structures are at least partially submerged therein, wherein a ratio of a combined surface area of the transparent structures contacted by the liquid to a volume of the liquid is between about 0.25 mm² per microliter and about 8.0 mm² per microliter.

3. An enzyme linked immunosorbent assay (ELISA) kit, comprising:

one or more reagents for an ELISA and a sensor assembly adapted for the ELISA, wherein the sensor assembly comprises:

a transparent container having one or more cavities formed therein,

a plurality of active surfaces disposed in each of the one or more cavities and configured for immobilizing a reagent thereon, and

one or more transparent detection structures disposed in each of the one or more cavities, wherein each of the transparent detection structures comprises one or more main surfaces that provide one or more of the active surfaces,

wherein each of the active surfaces is separated from an immediately adjacent one of the active surfaces by a distance between about 500 microns and about 8 mm.

4. An enzyme linked immunosorbent assay (ELISA) kit, comprising:

one or more reagents for an ELISA and a sensor assembly adapted for the ELISA, wherein the sensor assembly comprises:

a transparent container having one or more cavities formed therein,

a plurality of active surfaces disposed in each of the one or more cavities and configured for immobilizing a reagent thereon, and

one or more transparent detection structures disposed in each of the one or more cavities, wherein each of the transparent detection structures comprises one or more main surfaces that provide one or more of the active surfaces,

wherein at least one of the active surfaces comprises a textured polymeric surface having microstructures or nanostructures.

5. An enzyme linked immunosorbent assay (ELISA) kit, comprising:

one or more reagents for an ELISA and a sensor assembly adapted for the ELISA, wherein the sensor assembly comprises:

a transparent container having one or more cavities formed therein, and

one or more transparent detection structures disposed in each of the one or more cavities,

wherein inner surfaces of the cavities and main surfaces of the one or more transparent detection structures provide thereon active surfaces configured for immobilizing a reagent configured to specifically bind to an analyte, and

wherein the main surfaces of the transparent detection structures are configured such that, upon performing the ELISA, a detectable optical density corresponding to the analyte specifically bound to the immobilized reagent is increased without decreasing a rate of specifically binding the analyte to the immobilized reagents, relative to the sensor assembly without the one or more transparent detection structures.

6. The ELISA kit of any one of Embodiments 1 to 5, wherein each of the transparent detection structures comprises a transparent solid polymeric structure.

7. The ELISA kit of any one of Embodiments 1 to 6, wherein each of the active surfaces comprises a solid polymeric surface.

8. The ELISA kit of any one of Embodiments 1 to 7, wherein the active surfaces do not include a metal formed thereon.

9. The ELISA kit of any one of Embodiments 1 to 8, wherein the optically transparent container comprises a cuvette.

10. The ELISA kit of Embodiment 9, wherein each of the one or more transparent detection structures comprises a plate structure having opposing main surfaces that are substantially parallel to a depth direction of the cavity of the cuvette.

11. The ELISA kit of Embodiments 9 or 10, wherein each of the one or more transparent detection structures comprises a plate structure having opposing main surfaces that are substantially parallel to each other.

12. The ELISA kit of any one of Embodiments 9 to 11, wherein one or more of the main surfaces of the one of the one or more transparent detection structures directly facing inner sidewalls of the cuvette are separated from the inner sidewalls of the cuvette by the distance exceeding 500 microns.

13. The ELISA kit of any one of Embodiments 9 to 12, wherein one or more of the main surfaces of the one or more transparent detection structures directly facing inner sidewalls of the cuvette are substantially parallel to the inner sidewalls of the cuvette.

14. The ELISA kit of any one of Embodiments 9 to 13, comprising two or more transparent detection structures, wherein main surfaces of directly adjacent ones of the two or more transparent detection structures directly facing each other are separated from each other by the distance exceeding 500 microns.

15. The ELISA kit of any one of Embodiments 9 to 14, comprising two or more transparent detection structures, wherein main surfaces of directly adjacent ones of the two or more transparent detection structures directly facing each other are substantially parallel to each other.

16. The ELISA kit of any one of Embodiments 9 to 15, wherein one or more of the main surfaces of the one or more transparent detection structures and one or more of the inner sidewalls of the cuvette serve as the active surfaces.

17. The ELISA kit of any one of Embodiments 9 to 16, wherein one or more of main surfaces of the one or more transparent detection structures and the sidewalls of the cuvette substantially overlap one another in a lateral direction orthogonal to a depth direction of the cavity of the cuvette.

18. The ELISA kit of any one of Embodiments 9 to 17, wherein the one or more transparent detection structures have a thickness between about 100 microns and about 53-000 microns.

19. The ELISA kit of any one of Embodiments 9 to 18, wherein each of the main surfaces has an area between about 10 mm² and about 100 mm².

20. The ELISA kit of any one of Embodiments 9 to 19, wherein the cuvette is configured to hold a liquid having a volume between about 50 mm³ and about 3000 mm³.

21. The ELISA kit of any one of any one of Embodiments 1 to 8, wherein the optically transparent container comprises a strip container comprising a plurality of cavities formed therein.

22. The ELISA kit of Embodiment 21, wherein each of the one or more transparent detection structures comprises a plate structure having opposing main surfaces that are substantially perpendicular to a depth direction of the cavities.

23. The ELISA kit of Embodiments 21 or 22, wherein each of the one or more transparent detection structures comprises a plate structure having opposing main surfaces that are substantially parallel to each other.

24. The ELISA kit of any one of Embodiments 21 to 23, wherein a main surface of one of the one or more transparent detection structures directly facing a bottom surface of a respective one of the cavities is separated from the bottom surface of the respective one of the cavities by the distance exceeding 500 microns.

25. The ELISA kit of any one of Embodiments 21 to 24, wherein a main surface of one of the one or more transparent detection structures directly facing a bottom surface of a respective one of the cavities is substantially parallel to the bottom surface of the respective one of the cavities.

26. The ELISA kit of any one of Embodiments 21 to 25, wherein each of the one or more transparent detection structures comprises a plate structure disposed laterally at a central region of a respective one of the cavities.

27. The ELISA kit of any one of Embodiments 21 to 26, wherein one or more of the main surfaces of the one or more transparent detection structures substantially overlap one another in a vertical direction substantially parallel to a depth direction of the cavities.

28. The ELISA kit of any one of Embodiments 21 to 27, comprising two or more transparent detection structures, wherein main surfaces of directly adjacent ones of the two or more transparent detection structures directly facing each other are separated from each other by the distance exceeding 500 microns.

29. The ELISA kit of any one of Embodiments 21 to 28, comprising two or more transparent detection structures, wherein main surfaces of directly adjacent ones of the two or more transparent detection structures directly facing each other are substantially parallel to each other.

30. The ELISA kit of any one of Embodiments 21 to 29, wherein each of the transparent detection structures has a substantially circular shape.

31. The ELISA kit of any one of Embodiments 21 to 25, wherein each of the one or more transparent detection structures comprises a protrusion extending from an inner surface of the respective one of the cavities.

32. The ELISA kit of Embodiment 31, wherein each of the one or more transparent detection structures comprises a plate structure extending laterally towards a central region of the respective one of the cavities.

33. The ELISA kit of Embodiments 31 or 32, wherein the one or more transparent detection structures comprise one or more first protrusions formed at a first vertical level in a depth direction of the cavities.

34. The ELISA kit of Embodiment 33, wherein the one or more transparent detection structures comprise one or more second protrusions formed at a second vertical level in the depth direction of the cavities.

35. The ELISA kit of Embodiment 34, wherein at least one of the one or more first protrusions do not overlap any of the one or more second protrusions in any lateral direction perpendicular to a depth direction of the cavities.

36. The ELISA kit of Embodiment 34, wherein at least one of the one or more first protrusions at least partially overlap one or more second protrusions in a lateral direction perpendicular to a depth direction of the cavities.

37. The ELISA kit of any one of Embodiments 34 to 36, wherein the one or more transparent detection structures comprise two or more first protrusions and/or two or more second protrusions that are periodically arranged around the inner surface of the respective one of the cavities.

38. The ELISA kit of any one of Embodiments 34 to 37, wherein the one or more first protrusions and the one or more second protrusions are separated in a depth direction of the cavities by a spacer region that does not have protrusions.

39. The ELISA kit of any one of Embodiments 31 to 38, wherein one or more of the main surfaces of the one or more transparent detection structures and one or more of inner surfaces of respective ones of the cavities serve as the active surfaces.

40. The ELISA kit of any one of Embodiments 31 to 39, wherein a central region of each of the cavities that is unoccupied by the one or more transparent detection structures is configured to receive a tip of a pipette therein.

41. The ELISA kit of any one of Embodiments 21 to 38, wherein each of the one or more transparent detection structures has a shape comprising a portion of a circle.

42. The ELISA kit of any one of Embodiments 21 to 41, wherein the one or more transparent detection structures have a thickness between about 100 microns and about 2000 microns.

43. The ELISA kit of any one of Embodiments 21 to 42, wherein each of the main surfaces of the one or more transparent detection structures has an area between about 10 mm2 and about 40 mm2.

44. The ELISA kit of any one of Embodiments 21 to 43, wherein each of the cavities is configured to hold a liquid having a volume between about 50 mm³ and about 500 mm³.

45. The ELISA kit of Embodiment 31, wherein the one or more transparent detection structures comprise protrusions extending from an inner surface of respective ones of the cavities, wherein the protrusions form corrugations on inner surfaces of the cavities.

46. The ELISA kit of Embodiment 45, wherein the corrugations have a length extending through at least a partial depth of the cavities.

47. The ELISA kit of any one of Embodiments 1 to 46, wherein at least one of the active surfaces comprises a plurality of microstructures or nanostructures formed thereon.

48. The ELISA kit of Embodiment 47, wherein the microstructures or nanostructures comprise polymeric microstructures or nanostructures.

49. The ELISA kit of Embodiment 47 or 48, wherein the microstructures or nanostructures comprise regularly arranged nanostructures.

50. The ELISA kit of any one of Embodiments 47 to 49, wherein each of the microstructures or nanostructures comprises a protrusion having a cross sectional area that decreases away from a base.

51. The ELISA kit of any one of Embodiments 47 to 50, wherein the microstructures or nanostructures have a shape of a truncated sphere or polygon.

52. The ELISA kit of any one of Embodiments 47 to 50, wherein the microstructures or nanostructures have a prismatic shape.

53. The ELISA kit of any one of Embodiments 47 to 50, wherein the microstructures or nanostructures have a shape of a truncated cylinder.

54. The ELISA kit of any one of Embodiments 47 to 50, wherein the microstructures or nanostructures have a shape of a conical frustum or a pyramidal frustum.

55. The ELISA kit of Embodiments 47 or 48, wherein the microstructures or nanostructures are randomly or pseudo-randomly arranged in one or more lateral directions.

56. The ELISA kit of any one of Embodiments 47, 48 or 55, wherein the microstructures or nanostructures comprise microwires, micropillars, microfibers, nanowires, nanopillars or nanofibers.

57. The ELISA kit of any one of Embodiments 47 to 56, wherein the microstructures or nanostructures form integral extensions comprising the same material as a solid substrate.

58. The ELISA kit of any one of Embodiments 1 to 57, wherein the active surfaces of the sensor assembly has immobilized thereon a capturing reagent configured to specifically bind to a target analyte.

59. The ELISA kit of any one of Embodiments 1 to 58, wherein the one or more cavities has a solution comprising a target analyte and a marker-labeled detection reagent that is configured to specifically bind to a target analyte.

60. The ELISA kit of any one of Embodiments 1 to 59, wherein the one or more cavities has a solution comprising a target analyte bound to a marker-labeled detection reagent, and further comprises an enzyme substrate.

61. The ELISA kit of any one of Embodiments 1 to 60, wherein the one or more cavities has an ELISA product produced by an enzyme substrate in an ELISA reaction.

62. A method of conducting an enzyme linked immunosorbent assay (ELISA), the method, comprising;

providing an ELISA kit according to any one of Embodiments 1 to 61; and

conducting an ELISA reaction within the optically transparent container.

63. The method of Embodiment 62, wherein conducting the ELISA reaction comprises:

providing a solution comprising a target analyte and a marker-labeled detection reagent that is configured to specifically bind to the target analyte;

immobilizing on the active surfaces of the sensor assembly a capturing reagent configured to specifically bind to a target analyte;

at least partially immersing the active surfaces in the solution to cause the target analyte to be specifically bound to the capturing reagent and to the marker-labeled detection reagent; and

detecting the target analyte specifically bound to the capturing reagent and to the marker-labeled detection reagent.

64. The method of Embodiment 64, wherein the target analyte is specifically bound to the capturing reagent and to a marker-labeled detection reagent in a single reaction step in 30 minutes or less prior to detecting the target analyte specifically bound to the capturing reagent and to the marker-labeled detection reagent.

65. The method of Embodiments 63 or 64, further comprising washing the sensor assembly after causing the analyte to be specifically bound to the capturing reagent and to the marker-labeled detection reagent, wherein the method does not include additional washing steps.

66. The method of any one of Embodiments 62 to 65, wherein the ELISA is selected from the group consisting of: direct ELISA, indirect ELISA, sandwich ELISA, competitive ELISA and enzyme-linked immunoSpot (ELISPOT) assay.

67. A method of conducting an ELISA, the method comprising:

providing an ELISA well, wherein the ELISA well comprises:

1) a transparent container, and

2) more than one enhancement layer within the optically transparent container, wherein the more than one enhancement layer is configured to allow an antibody to be bound to it, wherein the more than one enhancement layer provides a ratio of a combined surface area of the more than one enhancement layer to a volume of the liquid is between about 0.25 mm² per microliter and about 8.0 mm² per microliter; and

conducting an ELISA with the optically transparent container, wherein only a single wash is involved in the ELISA.

68. The method of Embodiment 67, wherein the ELISA comprises: incubating a marker-labeled detection antibody with a sample containing a target protein so as to form a first reaction mixture.

69. The method of Embodiment 68, wherein the ELISA further comprises: taking the first reaction mixture and reacting it with a substrate immobilized with an immobilized antibody.

70. The method of Embodiment 69, wherein the ELISA can be performed by one-time addition of the sample.

71. Any one of the method based Embodiments provided herein, wherein the ELISA aspect comprises incubating HRP and adding a substrate solution that comprises a substrate, wherein the substrate is converted by HRP to a detectable form.

72. The method of Embodiment 70, wherein the detectable form comprises a color signal.

73. A biosensor comprising a detection structure in the shape of a plate having a first surface and a second surface opposite the first surface wherein an immobilized substance specifically binding to a target analyte is arranged on at least one of the first and second surfaces.

74. The biosensor according to Embodiment 73, wherein microstructures or nanostructures in the form of projections are formed on at least one of the first and second surfaces of the detection structure and are attached with the immobilized substance on the outer surface thereof.

75. The biosensor according to Embodiment 73, wherein the target analyte is selected from the group consisting of amino acids, peptides, polypeptides, proteins, glycoproteins, lipoproteins, nucleosides, nucleotides, oligonucleotides, nucleic acids, sugars, carbohydrates, oligosaccharides, polysaccharides, fatty acids, lipid, hormones, metabolites, cytokines, chemokines, receptors, neurotransmitters, antigens, allergens, antibodies, substrates, cofactors, inhibitors, drugs, pharmaceuticals, nutrients, prions, toxins, poisons, explosives, pesticides, chemical warfare agents, biohazardous agents, bacteria, viruses, radioisotopes, vitamins, heterocyclic aromatic compounds, carcinogens, mutagens, narcotics, amphetamines, barbiturates, hallucinogens, waste products, contaminants, and mixtures thereof.

76. The biosensor according to Embodiment 73, wherein the detection structure is inserted into and immersed in a cuvette accommodating a sample containing the target analyte such that the immobilized substance reacts with the target analyte.

77. The biosensor according to Embodiment 76, further comprising a gripping member connected to one end of the detection structure and gripped by a user.

78. The biosensor according to Embodiment 77, further comprising a cap connecting the detection structure to the gripping member and releasably inserted into the inlet of the cuvette.

79. The biosensor according to Embodiment 78, further comprising a fixing member arranged on the outer surface of the cap and whose shape is changed to create resilience when the cap is inserted into the cuvette wherein the fixing member is brought into close contact with the inner circumferential surface of the cuvette by the resilience.

80. The biosensor according to Embodiment 76, wherein the detection structure is divided into an immersion portion immersed in the sample and a non-immersion portion having a narrow portion whose width is smaller than that of the immersion portion.

81. The biosensor according to Embodiment 80, wherein the narrow portion is recessed from at least one of both sides of the detection structure and extends along the lengthwise direction of the detection structure.

82. The biosensor according to Embodiment 76, wherein the detection structure is provided in plurality and the detection structures are spaced apart from and parallel to each other.

83. The biosensor according to Embodiment 73, further comprising at least one sensor strip comprising a body with a predetermined length and a plurality of reaction chambers recessed from one surface of the body to accommodate a sample containing the target analyte wherein the detection structure is arranged in each of the reaction chambers.

84. The biosensor according to Embodiment 83, further comprising a fixing plate having a surface to which the sensor strip is detachably attached.

85. The biosensor according to Embodiment 83, wherein the detection structure is provided in plurality and the detection structures are vertically spaced apart from each other.

86. The biosensor according to Embodiment 83, further comprising sample injection holes recessed from one surface of the body so as to be in communication with the reaction chambers.

87. The biosensor according to Embodiment 84, further comprising an insertion protrusion protruding from one surface of the fixing plate wherein the body is recessed or perforated to form an insertion recess into which the insertion protrusion is inserted such that the sensor strip is attached to the fixing plate.

88. The biosensor according to Embodiment 87, further comprising a fixing protrusion spaced from the insertion protrusion and protruding from one surface of the fixing plate such that the insertion protrusion comes into contact with an inwardly recessed corner of one end of the body when inserted into the insertion hole.

89. A sensor assembly adapted for an enzyme linked immunosorbent assay (ELISA), the sensor assembly comprising:

a sensor strip comprising one or more wells formed therein, each of the one or more wells having a sidewall and a bottom surface; and

one or more detection structures connected to the sidewall of each of the one or more wells,

wherein the one or more detection structures are configured to immobilize a biomolecule directly thereon.

90. The sensor assembly of Embodiment 89, further comprising the biomolecule immobilized directly on the one or more detection structures.

91. The sensor assembly of Embodiments 89 or 90, wherein the biomolecule comprises an antibody configured to specifically bind to a target analyte of the ELISA.

92. The sensor assembly of any one of Embodiments 89-91, wherein each of the one or more detection structures extend laterally towards a central region of a respective one of the one or more wells.

93. The sensor assembly of any one of Embodiments 89-92, wherein the one or more detection structures comprise one or more first detection structures formed at a first vertical level in a depth direction of the one or more wells.

94. The sensor assembly of any one of Embodiments 89-93, wherein the one or more detection structures comprise one or more second detection structures formed at a second vertical level deeper than the first depth in the depth direction.

95. The sensor assembly of any one of Embodiments 89-94, wherein at least portions of the one or more first detection structures do not overlap the one or more second detection structures in the depth direction of the one or more wells.

96. The sensor assembly of any one of Embodiments 89-95, wherein the sensor strip comprises a plurality of layers each having an opening formed therethrough, wherein at least one of the layers comprises the one or more detection structures protruding from a sidewall of the opening.

97. The sensor assembly of assembly of any one of Embodiments 89-96, wherein the sensor strip comprises at least two layers comprising one or more detection structures that are vertically separated by a spacer layer.

98. The sensor assembly of assembly of any one of Embodiments 89-97, wherein the one or more detection structures comprise corrugations protruding laterally towards a central region a respective one of the one or more wells and vertically elongated in the depth of the respective one of the one or more wells.

99. The sensor assembly of any one of Embodiments 89-99, wherein the one or more detection structures comprise a surface that is textured to have a plurality of microstructures or nanostructures formed thereon.

100. The sensor assembly of any one of Embodiments 89-99, wherein each of the one or more detection structures comprises a plate structure disposed laterally at a central region of a respective one of the one or more wells.

101. The sensor assembly of any one of Embodiments 89-100, wherein the one or more detection structures comprise at least two substantially parallel plate structures that substantially overlap one another in a depth direction of the one or more wells.

102. The sensor assembly of any one of Embodiments 89-101, wherein vertically adjacent ones of the at least two plate structures are separated from each other by the distance between 500 microns and 8 mm in the depth direction of the one or more wells.

103. The sensor assembly of any one of Embodiments 89-102, wherein the one or more wells are cylindrical wells having a planar bottom surface.

104. The sensor assembly of any one of Embodiments 89-103, wherein each of the wells are configured to hold a sample having a volume of about 50 μL to about 500 μL.

105. The sensor assembly of any one of Embodiments 89-104, wherein the one or more wells have a diameter between about 2 mm and about 9 mm.

106. The sensor assembly of any one of Embodiments 89-105, wherein when the one or more wells are filled with a sample, a ratio of a combined surface area contacted by the sample to a volume of the sample exceeds about 0.25 mm² per microliter.

107. A sensor assembly adapted for an enzyme linked immunosorbent assay (ELISA), the sensor assembly comprising:

a cuvette comprising a cavity;

a cap configured to close the cavity; and

one or more detection structures connected to the cap and configured to be at least partly immersed in a liquid sample when present in the cavity, wherein the one or more detection structures are configured to immobilize a biomolecule directly thereon.

108. The sensor assembly of Embodiment 107, further comprising the biomolecule immobilized directly on the one or more detection structures.

109. The sensor assembly of Embodiments 107 or 108, wherein the biomolecule comprises an antibody configured to specifically bind to a target analyte of the ELISA.

110. The sensor assembly of any one of Embodiments 107-109, wherein each of the one or more detection structures comprises a plate structure having opposing main surfaces that are substantially parallel to a depth direction of the cavity of the cuvette.

111. The sensor assembly of any one of Embodiments 107-110, wherein each of the one or more detection structures comprises a plate structure having opposing main surfaces that are substantially parallel to each other.

112. The sensor assembly of any one of Embodiments 107-111, comprising two or more detection structures, wherein main surfaces of directly adjacent ones of the two or more detection structures directly facing each other are separated from each other by the distance between about 500 microns and 9 mm.

113. The sensor assembly any one of Embodiments 107-112, comprising two or more detection structures, wherein main surfaces of directly adjacent ones of the two or more detection structures directly facing each other are substantially parallel to each other.

114. The sensor assembly of any one of Embodiments 107-113, wherein each of the one or more detection structures comprises a plate structure having straight edges extending in a depth direction of the cavity, wherein the straight edges comprise one or more recessed regions reducing a width of the plate structure.

115. The sensor assembly of any one of Embodiments 107-114, wherein at least one of the one or more detection structures comprise a surface that is textured to have a plurality of microstructures or nanostructures formed thereon.

116. The sensor assembly of any one of Embodiments 107-115, wherein when the cavity is filled with a sample, a ratio of a combined surface area contacted by the sample to a volume of the sample is about 0.1-8 mm² per microliter.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number, respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or whether these features, elements and/or states are included or are to be performed in any particular embodiment.

While certain embodiments have been described, these embodiments have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while features are presented in a given arrangement, alternative embodiments may perform similar functionalities with different components and/or sensor topologies, and some features may be deleted, moved, added, subdivided, combined, and/or modified. Each of these features may be implemented in a variety of different ways. Any suitable combination of the elements and acts of the various embodiments described above can be combined to provide further embodiments. The various features and processes described above may be implemented independently of one another, or may be combined in various ways. All possible combinations and subcombinations of features of this disclosure are intended to fall within the scope of this disclosure.

What is claimed is:

1. A sensor assembly adapted for an enzyme linked immunosorbent assay (ELISA), the sensor assembly comprising:
a sensor strip comprising one or more wells formed therein, each of the one or more wells having a sidewall and a bottom surface; and
one or more detection structures connected to the sidewall of each of the one or more wells,
wherein the one or more detection structures are configured to immobilize a biomolecule directly thereon.

2. The sensor assembly of claim 1, further comprising the biomolecule immobilized directly on the one or more detection structures.

3. The sensor assembly of claim 2, wherein the biomolecule comprises an antibody configured to specifically bind to a target analyte of the ELISA.

4. The sensor assembly of claim 1, wherein each of the one or more detection structures extend laterally towards a central region of a respective one of the one or more wells.

5. The sensor assembly of claim 1, wherein the one or more detection structures comprise one or more first detection structures formed at a first vertical level in a depth direction of the one or more wells.

6. The sensor assembly of claim 5, wherein the one or more detection structures comprise one or more second detection structures formed at a second vertical level deeper than the first depth in the depth direction.

7. The sensor assembly of claim 6, wherein at least portions of the one or more first detection structures do not overlap the one or more second detection structures in the depth direction of the one or more wells.

8. The sensor assembly of claim 1, wherein the sensor strip comprises a plurality of layers each having an opening formed therethrough, wherein at least one of the layers comprises the one or more detection structures protruding from a sidewall of the opening.

9. The sensor assembly of claim 8, wherein the sensor strip comprises at least two layers comprising one or more detection structures that are vertically separated by a spacer layer.

10. The sensor assembly of claim 1, wherein the one or more detection structures comprise corrugations protruding laterally towards a central region of a respective one of the one or more wells and vertically elongated in the depth of the respective one of the one or more wells.

11. The sensor assembly of claim 1, wherein the one or more detection structures comprise a surface that is textured to have a plurality of microstructures or nanostructures formed thereon.

12. The sensor assembly of claim 1, wherein each of the one or more detection structures comprises a plate structure disposed laterally at a central region of a respective one of the one or more wells.

13. The sensor assembly of claim 1, wherein the one or more detection structures comprise at least two substantially parallel plate structures that substantially overlap one another in a depth direction of the one or more wells.

14. The sensor assembly of claim 13, wherein vertically adjacent ones of the at least two plate structures are separated from each other by the distance between 500 microns and 8 mm in the depth direction of the one or more wells.

15. The sensor assembly of claim 1, wherein the one or more wells are cylindrical wells having a planar bottom surface.

16. The sensor assembly of claim 15, wherein each of the wells are configured to hold a sample having a volume of about 50 μL to about 500 μL.

17. The sensor assembly of claim 15, wherein the one or more wells have a diameter between about 2 mm and about 9 mm.

18. The sensor assembly of claim 1, wherein when the one or more wells are filled with a sample such that the one or more detection structures are immersed in the sample, a ratio of a combined surface area contacted by the sample to a volume of the sample is about 0.25 mm²/μL to about 8 mm²/μL.

19. A sensor assembly adapted for an enzyme linked immunosorbent assay (ELISA), the sensor assembly comprising:
a cuvette comprising a cavity;
a cap configured to close the cavity; and
one or more detection structures connected to the cap and configured to be at least partly immersed in a liquid sample when present in the cavity,
wherein the one or more detection structures are configured to immobilize a biomolecule directly thereon.

20. The sensor assembly of claim 19, further comprising the biomolecule immobilized directly on the one or more detection structures.

21. The sensor assembly of claim 19, wherein the biomolecule comprises an antibody configured to specifically bind to a target analyte of the ELISA.

22. The sensor assembly of claim 19, wherein each of the one or more detection structures comprises a plate structure having opposing main surfaces that are substantially parallel to a depth direction of the cavity of the cuvette.

23. The sensor assembly of claim 19, wherein each of the one or more detection structures comprises a plate structure having opposing main surfaces that are substantially parallel to each other.

24. The sensor assembly of claim 19, comprising two or more detection structures, wherein main surfaces of directly adjacent ones of the two or more detection structures directly facing each other are separated from each other by the distance between about 500 microns and 8 mm.

25. The sensor assembly of claim 19, comprising two or more detection structures, wherein main surfaces of directly adjacent ones of the two or more detection structures directly facing each other are substantially parallel to each other.

26. The sensor assembly of claim 19, wherein each of the one or more detection structures comprises a plate structure having straight edges extending in a depth direction of the cavity, wherein the straight edges comprise one or more recessed regions reducing a width of the plate structure.

27. The sensor assembly of claim 19, wherein at least one of the one or more detection structures comprise a surface that is textured to have a plurality of microstructures or nanostructures formed thereon.
The sensor assembly of claim 19, wherein the cuvette is configured to hold a liquid having a volume between about 50 mm³ and about 3000 mm³.

28. The sensor assembly of claim 19, wherein when the cavity is filled with a sample such that the one or more detection structures are immersed in the sample, a ratio of a combined surface area contacted by the sample to a volume of the sample is about 0.1 mm²/μL to about 8 mm²/μL.
